(19) United States
(12) Patent Application Publication
(10) Pub. No.: US 2002/0055124 A1
(43) Pub. Date: May 9, 2002

(54) Soluble support for organic synthesis

(75) Inventors: Kim D. Janda, La Jolla, CA (US); Dennis J. Gravert, Winona, MN (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolla, CA

(21) Appl. No.: 09/996,402

(22) Filed: Nov. 19, 2001

Related U.S. Application Data
(63) Continuation of application No. 09/161,604, filed on Sep. 23, 1998.

(51) Int. Cl. G01N 33/53; G01N 33/543; C08F 8/30; C08F 8/44; C08F 8/32

(52) U.S. Cl. 435/7.1; 436/518; 525/374; 525/375

(57) ABSTRACT

Polymer supports for liquid-phase organic synthesis (LPOS) are employed in a process for transferring a chemical intermediate between immiscilbe solvents. These compounds are produced with an expanded range of solubility range in a variety of solvent systems. A sequence of normal and “living” free radical polymerizations are employed to generate a library of block copolymers possessing either block or graft architecture with initiators 100, 200 and 3-4 and a diverse set of vinyl monomers 5-9. Novel bifunctional initiators are employed that have functional groups that independently produce free radical polymerizations to produce the block copolymers. Preferred functional groups include diazene (—N═N—) and 2,2,6,6-tetramethylpiperidinyl-1-oxy (TEMPO) moieties tethered by ester or ether linkages.

### TECHNICAL FIELD

The present invention relates to methods of organic synthesis. More particularly, the present invention relates to processes which employ soluble supports for transferring chemical intermediates between immiscible solvents.

### BACKGROUND

What is needed is a process and soluble polymer supports for transferring a chemical intermediate between immiscible solvents. What is needed is a continuum of rapidly generated soluble polymer supports adapted to achieve such transfer by conforming to the required solvent conditions, including the entire spectrum of organic and aqueous solution chemistry, which include positions amenable for chemical derivatization and functionalization.

### SUMMARY OF THE INVENTION

The invention is generally directed to a process for transferring a chemical intermediate from a first solvent to a second solvent and then from said second solvent to said first solvent, the first and second solvents being immiscible with one another. The invention is further generally directed to the synthesis of compounds which act as first and second conjugates (block copolymers) to facilitate the transfer of said chemical intermediates to the different solvent conditions. Also, the invention is generally directed to the synthesis of novel bifunctional initiators which have been designed with functional groups that independently produce free radical polymerizations to produce block copolymers. These initiators are synthesized to contain both diazene (—N═N—) and 2,2,6,6-tetramethylpiperidinyl-1-oxy (TEMPO) moieties tethered by ester or ether linkages.

More particularly, one aspect of the invention is directed to a process for transferring a chemical intermediate from a first solvent to a second solvent, the first and second solvents being immiscible with one another. A first conjugate is provided which serves as a solute in the first solvent. The first conjugate includes the chemical intermediate, a platform, and a first carrier. The chemical intermediate and the first carrier are attached to the platform. The first carrier has a solubility in the first solvent for imparting solubility to the first conjugate in the first solvent. The first conjugate is then converted into a second conjugate. The second conjugate includes the first conjugate and a second carrier. The second carrier is attached to the platform and has sufficient solubility in the second solvent to impart solubility to the entire second conjugate in the second solvent. The second conjugate is then contacted with the second solvent for transferring the second conjugate together with the chemical intermediate attached thereto from the first solvent into the second solvent.

Another aspect of the invention is directed to a continuation of the above process wherein the chemical intermediate is then transferred from the second solvent back to the first solvent. A second conjugate is provided which serves as a solute in the second solvent. The second conjugate includes the chemical intermediate, a platform, a first carrier, and a second carrier. The chemical intermediate, the first carrier and the second carrier are each attached to the platform. The second carrier has a sufficient solubility in the second solvent to impart solubility to the entire second conjugate in the second solvent. The second carrier is then cleaved from the platform of the second conjugate for transferring the chemical intermediate from the second solvent to the first solvent by forming a first conjugate having a solubility in the first solvent.

Another aspect of the invention is directed to a process for transferring a chemical intermediate into a first solvent. Firstly, the chemical intermediate and a first carrier are conjugated to a platform for forming a first conjugate. The first conjugate is then contacted with the first solvent for transferring the first conjugate together with first chemical intermediate attached thereto into the first solvent.

Another aspect of the invention is directed to a process for converting a first chemical intermediate into a second and third chemical intermediate. Firstly, a first conjugate is provided which serves as a solute in the first solvent. The first conjugate includes the first chemical intermediate, a platform, and a first carrier. The chemical intermediate and the first carrier are attached to the platform. The first carrier has sufficient solubility in the first solvent for imparting solubility to the entire first conjugate in the first solvent. The first chemical intermediate, while attached to the first conjugate, is then converted into the second chemical intermediate in the first solvent, thereby forming a modified first conjugate. The modified first conjugate having the second chemical intermediate attached thereto is then converted into a second conjugate. The second conjugate includes the second chemical intermediate, the platform, the first carrier, and the second carrier. The second chemical intermediate and the first and second carriers are attached to the platform. The second carrier has sufficient solubility in the second solvent to impart solubility to the entire second conjugate in the second solvent. The second conjugate is then contacted with the second solvent for transferring the second conjugate together with the second chemical intermediate attached thereto into the second solvent. The second chemical intermediate, while attached to the second conjugate, is then converted into the third chemical intermediate in the second solvent for forming a modified second conjugate.

Another aspect of the invention is directed to a process for synthesizing a first conjugate. The first conjugate includes a platform and a first carrier attached to one another. Firstly, a bifunctional initiator and the first carrier are provided. Heat is then applied to the bifunctional initiator and the first carrier. The application of heat causes the bifunctional initiator to convert into the platform and the first carrier to attach to the platform for synthesizing the first conjugate.

Another aspect of the invention is directed to a process for synthesizing a second conjugate. The second conjugate includes a platform with a first and second carrier attached thereto. Firstly, a first conjugate is provided as a solute in a first solvent. The first conjugate includes a platform with a first carrier attached thereto. The first carrier is attached to the platform. The first carrier has sufficient solubility in the first solvent for imparting solubility to the first conjugate in the first solvent. The first conjugate is then converted into a second conjugate. The second conjugate includes the first conjugate and a second carrier. The second carrier is attached to the platform and has sufficient solubility in a second solvent to impart solubility to the second conjugate in the second solvent.

Another aspect of the invention is directed to a solution having a first solvent and a first conjugate mixed as a solute therein. The first conjugate includes a chemical intermediate, a platform, and a first carrier. The chemical intermediate and the first carrier are attached to the platform. In a preferred embodiment, the first conjugate may be represented by the following structure:

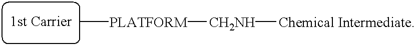

An exemplary first conjugate may be represented by the following structure:

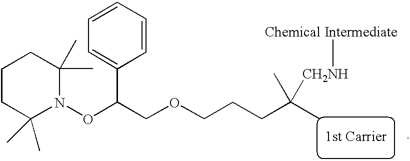

Another exemplary first conjugate may be represented by the following structure:

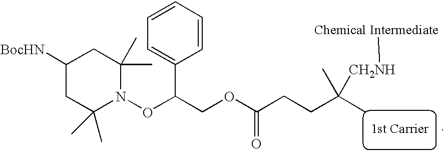

Another aspect of the invention is directed to a solution having a second solvent and a second conjugate mixed as a solute therein. The second conjugate includes a platform with a chemical intermediate and a first and second carrier attached thereto. In a preferred embodiment, the second conjugate represented by the following structure:

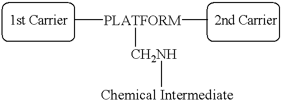

An exemplary second conjugate may be represented by the following structure:

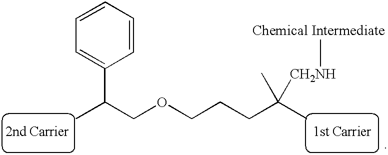

Another exemplary second conjugate may be represented by the following structure:

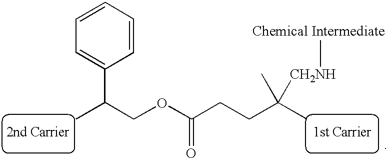

Another aspect of the invention is directed to a solution having a first solvent and a first conjugate mixed as a solute therein. The first conjugate includes platform with a chemical intermediate and a plurality of first carriers attached thereto.

Another aspect of the invention is directed to advanced intermediate. Exemplary advanced intermediates may be represented by the following structures:

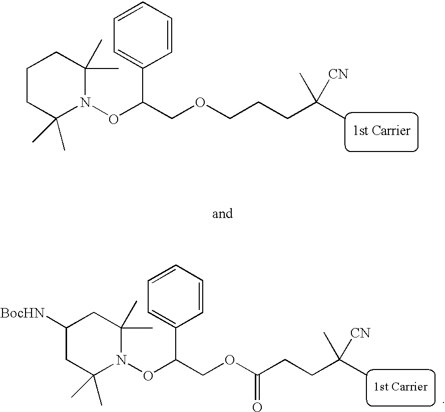

Another aspect of the invention is directed to bifunctional initiator. Exemplary bifunctional initiators may be represented by the following structures:

[chemical structure]

Another aspect of the invention is directed to a conjugate molecule represented by the following structure:

[chemical structure]

### DESCRIPTION OF FIGURES

FIG. 1 shows free radical initiators 100, 200 and 3-4 and vinylic monomers 5-9 utilized for copolymer (1st and 2nd conjugate) library construction.

FIG. 2 illustrates the sequence of normal/“living” polymerization with initiators 100, 200 and 3-4 and 1st carrier and 2nd carrier showing the potential architecture of the block and graft copolymer (conjugate) products and the end groups for derivatization.

FIG. 3 illustrates the synthetic route to the radical initiators 3 and 4.

FIG. 4 illustrates the reduction of the a-nitrile groups in polyBS-DS to give poly 22 and subsequent kinetic evaluation of imine formation with 23.

FIG. 5 illustrates the synthetic route to polymer supported phosphine ligand 27 and subsequent catalytic reduction of 28 to 29.

FIG. 6 shows an outline of the ‘oscillating liquid phase strategy’ showing the potential for changing polymer support solubility from organic to aqueous to organic with an organic polymer block—A and an aqueous polymer block—B and vice versa. Block copolymers polyBA-AA and polyBS-VP have been investigated for their utility in this approach.

FIG. 7 illustrates block copolymerization using bifunctional free radical initiators.

FIG. 8 illustrates the synthesis of compound 100 using the procedures as described in the experimental protocols and the following conditions:

a, 1 equiv. 3, 15 equiv. 4, 3 equiv. 5, <30° C., 2 h, then 50° C., 16 h, 38%; b, 12 equiv. NaOH, THF/MeOH/water (3:1:1), 20° C., 16 h, 89%; c, 2.05 equiv. 7, 1 equiv. 8, 3.6 equiv. EDC, 3.6 equiv. HOBT, DMF, 20° C., 72 h, 65%.

FIG. 9 illustrates the synthesis of compound 200 using the procedures as described in the experimental protocols and the following conditions: a, 1.9 equiv. 9, 1 equiv. hydrazine sulfate, 2 equiv. NaCN, water, ₂0° C., 72 h, then excess conc. HCl followed by 1 equiv. Br2 over 4 h, 0° C., 29%; b, 3 equiv. MsCl, 3 equiv. NEt3, CH₂Cl₂, 20° C., 2 h, 95%; c, 2.1 equiv. 7, 2.06 equiv. KH, 2.1 equiv. DMSO, THF, 10 min, 20° C., then transferred to 11 in THF, 90 min, 20° C., 64%.

FIG. 10 shows a table which outlines the solubility of the block copolymer library members from initiator 200 with the following notations: a) Monomers: styrene 5 (S), 4-tert -buty1styrene 6 (BS), 3,4-dimethoxystyrene 7 (DS), N -vinylpyrrolidinone 8 (VP), N-isopropylacrylamide 9 (IA). b) Solvents: toluene (A), diethyl ether (B), tetrahydrofuran (C), acetone (D), acetonitrile (E), dichloromethane (F), dimethylformamide (G), dimethylsulfoxide (H), methanol (I), water (J). c) S soluble; sw=swell; I=insoluble.

FIG. 11 shows a table which outlines the physical properties of the block copolymer library derived from initiator 200 with the following notations: a) Mn and PD measured on three Styrogel (Waters) columns in series (7.8×300 mm: 104 Å, 103 Å, 500 Å) calibrated with ten monodisperse (Mw/Mn<1.13) polystyrene standards (Mn: 3.15×106, 1.29×106, 6.30×105, 1.71×105, 6.60×104, 2.85×104, 1.29×104, 5.46×103, 1.70×103, 580). b) SEC mobile phase solvent: A=THF, B=CHCl₃. c) Molar ratio of second monomer in the copolymer as measured by 1H NMR. d) Yield calculated from the theoretical yield (see experimental section) e) Not applicable: homopolymer.

FIG. 12 shows a table which outlines the solubility of graft copolymer library from initiator 4 with the following notations: a) Monomers: styrene 5 (S) 3,4-dimethoxystyrene 7 (DS), N-vinylpyrrolidinone 8 (VP). b) Solvents: toluene (A), diethyl ether (B), tetrahydrofuran (C), acetone (D), acetonitrile (E), dichloromethane (F), dimethylformamide (G), dimethylsulfoxide (H), methanol (I), water (J). c) S=soluble; sw=swell; I=insoluble.

### DETAILED DESCRIPTION OF THE INVENTION

The present invention relates to a process for transferring a chemical intermediate from a first solvent to a second solvent and then from said second solvent to said first solvent, the first and second solvents being immiscible with one another. A further aspect of the invention relates to the synthesis of compounds which act as first and second conjugates (block copylmers) to facilitate the transfer of said chemical intermediates to the different solvent conditions. Another aspect of the invention is directed to the synthesis of novel bifunctional initiators which have been designed with functional groups that independently produce free radical polymerizations to produce block copolymers. These initiators are synthesized to contain both diazene (—N═N—) and 2,2,6,6-tetramethylpiperidinyl-1-oxy (TEMPO) moieties tethered by ester or ether linkages.

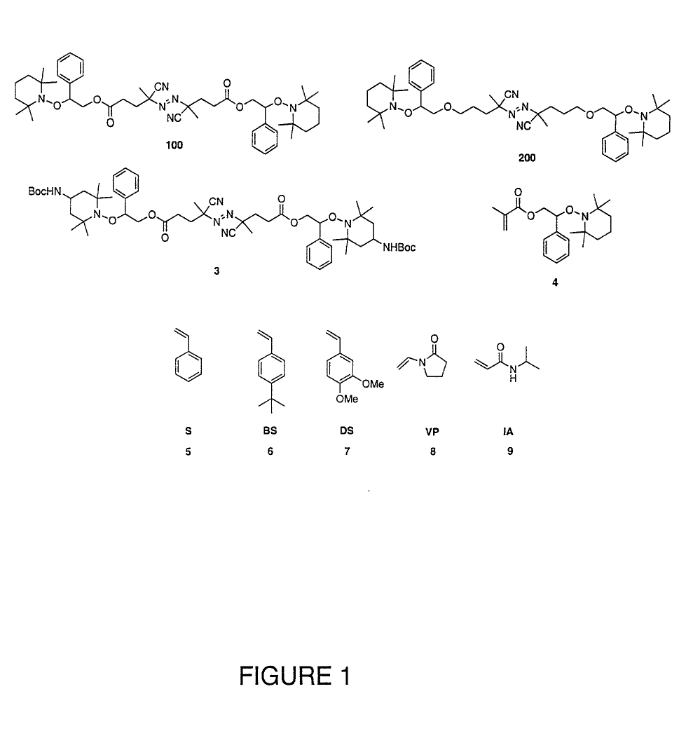

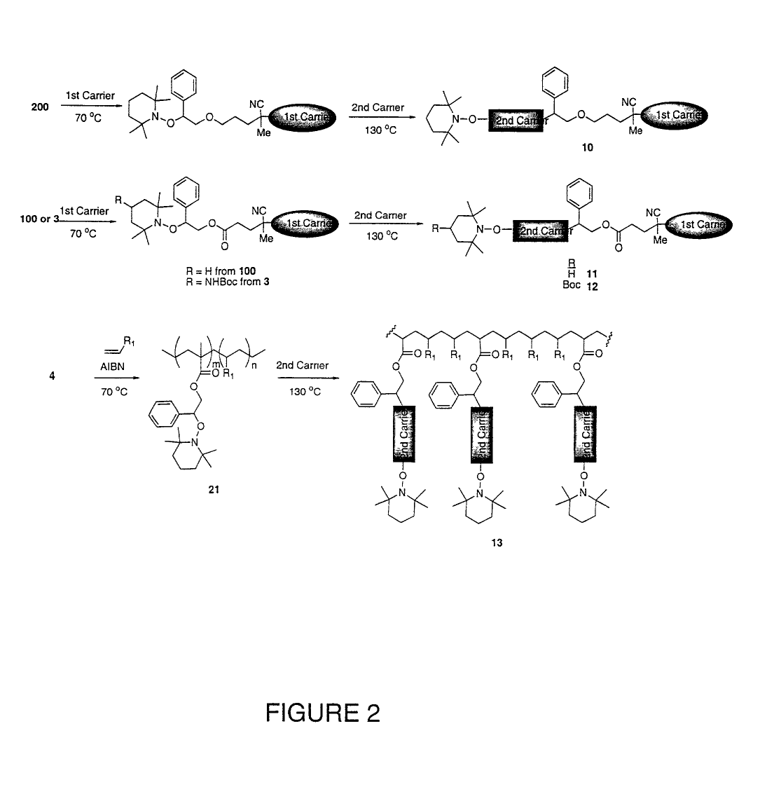

Another aspect of the invention is directed to the utilization of a sequence of normal and living free radical processes with initiators as exemplified through compounds 100, 200, 3-4 and monomers (carriers) 5-9 to generate a parallel array of either block (derived from bifunctional initiators 100, 200 and 3) or graft (derived from initiator 4) copolymers which exhibit unique solubility profiles (FIG. 1). These new block (class 10-12) or graft (class 13) copolymers, by virtue of the structural remnants of the initiators that generated them, contain loci that are amenable for derivatization and as discussed vide infra makes them of ultimate value in LPOS (FIG. 2).

Libraries of block and graft copolymers have been generated by a sequence of normal and “living” free radical polymerization with a variety of vinyl monomers 5-9 and initiators 100,200 and 3-4. One block copolymer selected from these libraries, polyBS-DS, has a solubility profile that is complementary to the current soluble polymer of choice in LPOS, PEG, and hence may be even more useful when applied in soluble polymer organic synthesis. Hydrolysis of the terminal TEMPO residues of the copolymers to generate hydroxyl residues has proven to be difficult by standard methodologies. However the α-nitrile groups of polyBS-DS are faciley reduced with LiAlH₄ or PtO2/H₂. Kinetic studies have revealed that the accessibility of these amino functionalities for reaction is essentially equivalent to a small molecule in solution. As an example of polyBS-DS in LPOS, a rhodium(I) phosphine polyBS-DS complex Rh(I)-27 catalyzes the asymmetric reduction of 2N-acetylacrylic acid 28 at the same rate and with a similar optical yield to a rhodium(I)-phosphine ligand in solution.

Other embodiments of the invetion comprise copolymers polyBS-DS-(NBoc) and polyVP-S-(NBoc) which are derived from inititator 3 possess TEMPO end groups functionalized with Boc protected amino residues which can be easily hydrolyzed with a TFA/DCM (1:10) mixture to incorporate additional sites for derivatization. In addition an “oscillating liquid-phase” strategy can be performed (with polymers derived from initiators 100, and 3-4) where the solubility of the copolymer support can be modified during a synthetic strategy by either a second polymerization during a synthetic scheme or hydrolysis of the ester linkage between the blocks to free the component homopolymer fragments.

Finally, the adaptability of these new soluble polymer supports makes them ideal for additional applications in high -throughput organic synthesis such as potential fluorous phase compatibility and offers soluble polymer analogs to resin-capture, polymer-quench, and complementary molecular recognition (CMR) strategies (Keating et al. J. Am. Chem. Soc. 1996, 118, 2574; Booth et al. J. Am. Chem. Soc. 1997, 119, 4882; Flynn et al. J. Am. Chem. Soc. 1997, 119, 4874).

### EXAMPLE 1

### Soluble Supports Tailored for Organic Synthesis: Parallel Polymer Synthesis via Sequential Normal/Living Free Radical Processes

Bifunctional Initiator Design and Synthesis: Having settled upon a radical polymerization strategy, the next stage was the design and synthesis of suitable initiators. Diazene and TEMPO moieties are known to initiate/mediate free radical polymerization at 70° C. and 130° C., respectively. Therefore we have synthesized bifunctional free radical initiators 100, 200 and 3 (FIG. 2; Li et al. Macromolecules 1997, 30, 5195; Graver et al. Tetrahedron Lett. 1998, 39, 1513), which contain an a-nitrile diazene core (—N═N—) linked via a spacer to two TEMPO molecules. This inherent bifunctionality of 100, 200 and 3 is designed to provide for two independent rounds of polymerization, thus, block copolymers can be obtained in a temperature controlled manner through sequential normal and “living” polymerizations to give block copolymers of type 10-12 (FIG. 2). The synthesis of initiators 100 and 200 is described in example 2, below.

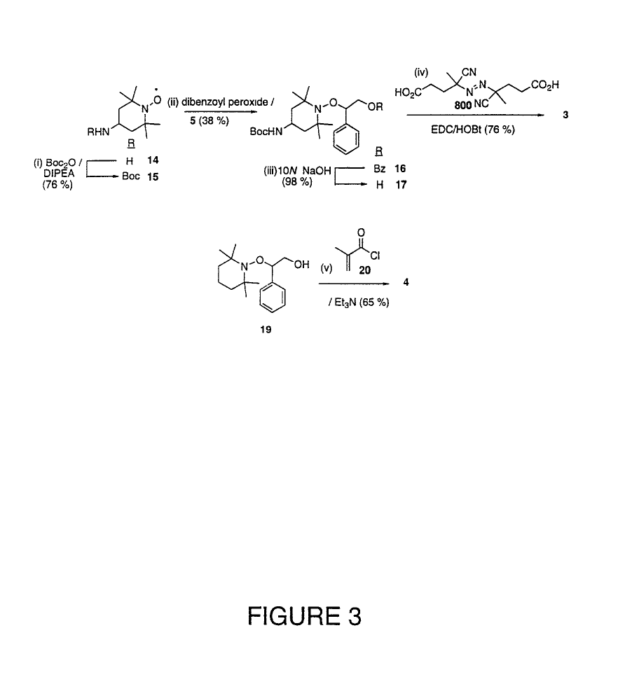

Initiator 3 was synthesized in four steps from commercially available 4-amino TEMPO 14 by an initial Boc protection to give the TEMPO derivative 15 in 76% yield (FIG. 3). The critical reaction of styrene 5, dibenzoyl peroxide and 15 then gave the key benzoyl protected derivative 16 in acceptable yield (38%) following silica gel chromatography. Saponification of 16 proceeded smoothly in a 10 N NaOH/MeOH/THF (3:1:1) mixture to give the TEMPO alcohol derivative 17 in 98% yield. The bis esterification of 4,4′-azobis(4-cyanovaleric acid) 800 with 17 occurred via EDC/HOBt coupling conditions in tetrahydrofuran (THF) to give initiator 3 in 76% yield.

As discussed vide supra sequential normal/“living” polymerizations can produce graft (or comb) copolymers in addition to block copolymers (FIG. 2). To expand the structural diversity of our copolymer library and to explore the solubility properties of comb polymers vs block polymers, TEMPO-functionalized methacrylate 4 was synthesized as the final initiator/mediator in our strategy via esterification of 1-hydroxy-2-phenyl-2-(2,2′,6,6′-tetramethyl-1-piperidinyloxy)ethane 19 with methacryloyl chloride 20 in good yield (65%) (FIG. 3). In contrast to initiators 100, 200, and 3, 4 participates as a monomer in the first polymerization with monomer A, resulting in statistical copolymers of type 21 (FIG. 2). The TEMPO functionalized residues then mediate the ′living′ polymerization at 130° C. with monomer B giving rise to comb copolymers of type 13.

Intrinsic in the structures of 100, 200, 3, and 4 is that the copolymers formed by the normal and “living” free radical sequence of polymerization, either di- or tri-block, will contain residues amenable for derivatization and hence be of use in LPOS (FIG. 2). The block copolymers 10-12 derived from initiators 100, 200 and 3 possess a-nitrile residues, which can be converted into amino groups by reduction. A structural feature common to all the classes of copolymers 10-13 formed by this strategy, a result of the known termination mechanism of “living” free radical polymerization, is that the end of the copolymer chains may possess TEMPO groups.

It is known that the TEMPO functionality can be removed under reductive conditions (Zn/acetic acid or Zn/NH₄Cl; Barrett et al. J. Org. Chem. 1990, 55, 5196; Shishido et al. J. Org. Chem. 1992, 57, 2876; Boger et al. J. Org. Chem. 1995, 60, 1271) to give terminal hydroxyl groups. Additionally, implicit in the design of initiator 3 is that the Boc group of the 4-amino TEMPO residues (class 12 copolymers) may be faciley removed with TFA hence generating a terminal amino group serving to increase the loading of these polymer supports.

The final component of the initiators' 100, 200, 3 and 4 design requiring discussion is the moiety that links the α-diazene core and the TEMPO end groups. For initiator 200 this is a dialkyl ether whereas in 100, 3-4 this is a substituted homobenzylic ester. Again it should be noted from FIG. 2, that these linkages become incorporated into the block or graft copolymers during the polymerization sequence which is an advancement of tandem “living” free radical polymerization methodology. Existing methods utilizing either ATRP or TEMPO produce block copolymers that do not provide linkages between polymeric blocks. While “link-functionalized” polymers (LFPs) have been synthesized using bis-initiators that link together two active polymerization centers to form both blocks simultaneously (Boffa et al. Macromolecules 1997, 30, 3494) our methodology provides for polymerization of each block independently using different monomers.

The incorporation of a chemically robust dialkyl ether linkage between the polymer blocks by initiator 200 was seen as fundamental for library construction of copolymers being considered for ultimate application in LPOS. Initiators 100, 3-4 were utilized when the lability of the ester linkage was to be exploited either during SEC analysis of polymer digests (to help confirm di- or tri-block structures) or during a process we have dubbed ‘oscillating liquid-phase’ (OLP) where the solubility of the polymer support can be modified during a synthetic strategy by saponification of the homobenzylic ester moiety thus fragmenting the copolymer into its constituent blocks.

Parallel Block Copolymer Synthesis Utilizing Initiator 200: Library synthesis occurred in a two-dimensional spatially addressable array format with five vinylic monomers S (5), BS (6), DS (7), VP (8), and IA (9). Polymerization reactions were conducted in a thick-walled reaction tube in a heated reactor/stirrer block affixed to an orbital shaker. Where possible the polymerizations were conducted neat, however in certain cases a minimum of solvent [dimethylformamide (DMF) or 1,2-dichlorobenzene (DCB)] was added to ensure homogeneous reaction conditions. Only the minimum amount of solvent was added because polydispersity (PD) has been reported to be directly proportional to the amount of solvent used in TEMPO-mediated polymerizations.

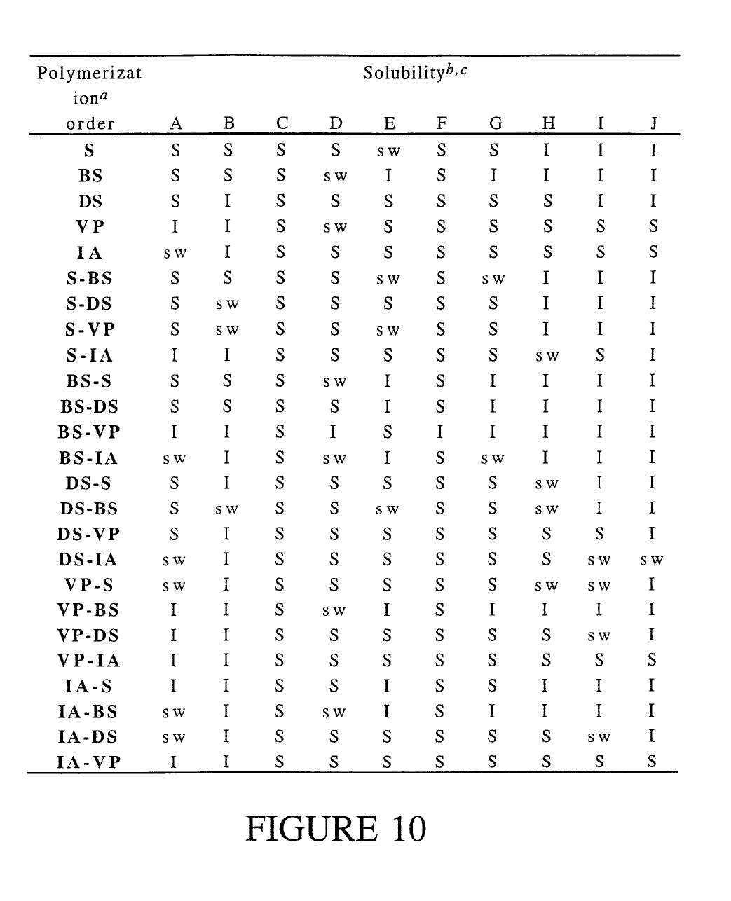

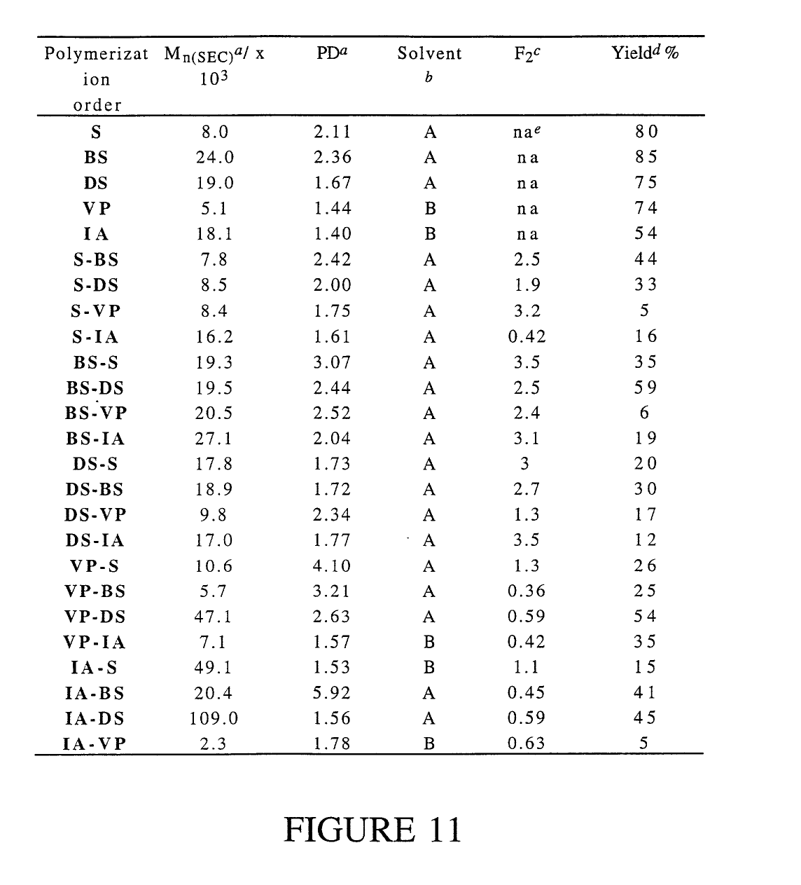

Following polymerization of initiator 200 with the first monomer (rigorously degassed by freeze thawing with liquid nitrogen) at 70 ° C. for 8-10 h, homopolymeric material was isolated from the reaction mixtures by precipitation with a suitable solvent. The resultant homopolymer was then dissolved in the second monomer, deoxygenated as described vide supra, and then was heated to 130 ° C. for 8-10 h. This library of twenty crude block copolymers (polymers of the 5×5 array containing all blocks of the same monomer were not synthesized) was isolated by precipitation following addition of suitable solvent mixtures. At this stage, selective solvents were used to remove unwanted homopolymer “impurities” from the isolated residues. In some cases however, such solvent systems could not be found and occasionally addition of selective solvents to crude polymeric products produced intractable suspensions that could not be easily filtered. However, it should be stressed that residual amounts of homopolymers produced as a result of chain transfer and/or termination events common to free radical polymerization approach, while elevating the value of the PDs observed, does not affect at all the ultimate use of these materials as soluble polymer supports in liquid-phase synthesis. Solubility characteristics and other physical properties of the polymer library are reported in the tables found in FIGS. 10 and 11.

Solubility properties of the twenty polymer supports were assayed in a panel of ten commonly used solvents (FIG. 10). Because solubility properties changed by linking together different polymer blocks, new supports were obtained that exhibited solubility profiles not matched by any other block copolymer or homopolymer studied. All polymer supports were soluble in tetrahydrofuran (THF) and dichloromethane (DCM). However, only copolymers containing blocks of both S and BS were soluble in diethyl ether (Et2O), while polymer supports based on blocks of two of the three polar monomers DS, VP, and IA were soluble in dimethylsulfoxide (DMSO). Polymerization of BS followed by VP yielded the block copolymer polyBS-VP which is insoluble in all solvents except THF, acetone, and DCM, but the copolymer formed from a first polymerization of DS followed by VP is soluble in all solvents studied except Et2O and water. In some cases, the solubility profiles of the block copolymers differed slightly between two polymer supports derived from the same monomers but polymerized in opposite order; however, these differences might also be attributable to differences in block lengths.

The only water soluble block copolymers contained blocks of both VP and IA. Hompolymers of VP and IA are both soluble in water, but upon heating above the cloud point of 31-32° C. polyIA precipitates (Heskins et al. J. Macromol. Sci. Chem. 1968, 1441). This inverse solubility behavior, characterized by a lower critical solution temperature (LCST), has been exploited previously to produce polymer supports that act as a temperature controlled switch for catalytic hydrogenation (Bergbreiter et al. J. Am. Chem. Soc. 1996, 118, 6092). Interestingly, aqueous solutions of polyVP-IA and polyIA-VP also clouded upon heating, with LCSTs measured at 38° C. and 35° C., respectively.

Characterization of all the copolymer library members by ¹H and ¹³C NMR spectroscopy gave results consistent with block copolymer structures. However, molecular weights measured by SEC [utilizing three Styrogel (Waters) columns in series] often did not increase significantly, from the homopolymer isolated from the first polymerization after the second polymerization as may be expected for block copolymerization. It should be stressed that SEC elution times can be influenced by polymer chemical composition (Handbook of Size Exclusion Chromatography, Wu, C. -S., Ed.; Marcel Dekker; New York, 1995; p 149) and discrepancies may result from molecular weight calculations of block copolymers based on their SEC elution times relative to polystyrene standards (Hawker et al. Macromolecules 1996, 29, 2686).

Even changing functional groups at polymer termini can lead to longer elution times and consequently, an apparently lower molecular weight value (Spychaj et al. Appl. Polym. Sci. Appl. Polym. Symp. 1991, 48, 199; Zhong et al. Macromolecules 1992, 25, 7160). In fact, we observed that between polystyrene samples produced by an anionic method (Mn=1700, PD=1.06, reported by Polymer Laboratories) and “living” radical polymerization (Mn=1000, PD=1.12, by SEC with THF),15 the order of elution from the SEC columns reversed upon changing the solvent from THF to chloroform (CHCl₃). Consequently with CHCl₃ as the mobile phase, the molecular weight of TEMPO-functionalized polystyrene was recalculated to be Mn=3200, PD =1.16 relative to the SEC elution times of the polystyrene standards. Thus, molecular weights calculated from data obtained from our SEC system serves only as an estimate of the true polymer chain lengths.

To help confirm the nature of the block copolymers synthesized by our strategy, a separate series of normal and “living” polymerizations were undertaken with the S monomer and initiator 100. Heating S and 100 at 70° C. overnight and precipitating the product, by addition of methanol (MeOH), yielded polys with an Mn=8200 and a PD=1.69. A sample of the polys homopolymer then was heated at 130° C. with additional S to produce polyS-S of higher molecular weight (Mn=264,000, PD=1.30). The ester bond between the polymer blocks (vide supra) was hydrolyzed with NaOH in a THF:MeOH:H₂O (3:1:1) mixture, and SEC analysis revealed complete loss of the peak of Mn=264,000 and concomitant formation of two peaks of Mn=118,000 (PD=1.22) and Mn=8700 (PD=1.44). Therefore, the measured molecular weight of 264,000 reported seems consistent with a triblock copolymer wherein two TEMPO-mediated blocks of Mn=118,000 are attached to one central diazene-initiated polystyrene block of Mn=8700. The peak assigned to the polystyrene block initiated by the diazene functionality (first block of Mn 8200) was not detected by SEC upon direct injection of the hydrolysis reaction (after removing water with Na2SO₄). Instead, only the TEMPO-mediated block (Mn=118,000; PD=1.22) was observed which was not unexpected as the block copolymer contained at a maximum 3.2% of the first block by weight. However, it was discovered that addition of ether to the hydrolysis reaction not only induced phase separation, but also caused the higher molecular weight polystyrene to partition out of the organic layer and collect at the interface as an emulsion. Thus, observation by SEC of the lower molecular weight polystyrene was achieved by concentrating the organic layer and adding only a small sample of emulsion found at the interface.

The formation of the triblock structure is most likely a consequence of head-to-head combination of two polymer chains during the first polymerization at 70° C. (Moad et al The Chemistry of Free Radical Polymerization; Pergamon; Oxford, 1995; p 228). This is the predominant mode of termination during normal free radical polymerization of S, however, other modes of termination are known to occur. In fact, the observed peak for polyS-S is asymmetric and suggests the presence of polymeric structures other than triblock. Chain transfer events and/or disproportionation that occur during diazene-initiated polymerization increase polydispersity and may lead to diblock, branched, or homopolymers following the second polymerization mediated by TEMPO. Ideally, such termination events should be absent in “living” radical polymerization however, homopolymer production via thermal initiation is a known side reaction during TEMPO-mediated polymerization.

Another piece of evidence suggesting that side reactions have occurred is given by the measured PD of 1.22 for the cleaved TEMPO-mediated block, as “living” radical polymerizations normally yield PDs <1.1. Finally, it should be pointed out that pathways of termination may differ for the monomers other than S and lead to polymeric structures with varying proportions of triblock, diblock and homopolymeric components.

For most applications in materials science, side reactions must be minimized to produce polymers with narrow molecular weight distributions. However, narrow PD is less important for polymer supports with ultimate use in organic synthesis. For example, a copolymer with PD=3.54 has been used successfully to prepare water-soluble, polymer-bound hydrogenation catalysts that were recovered by precipitation by alteration of pH. Of course there is a genuine concern that polymer supports with broad PD may suffer material losses of very short polymer chains which will remain in solution during the precipitation step, however, such low molecular weight polymers can be removed by performing several precipitations prior to using the polymer support for organic synthesis. In fact, this fractionation technique is a well known method for lowering PD (Polymer Fractionation, Cantow, M. J. R., Ed.; Academic Press; New York, 1967; Noshay et al. Block Polymers; Academic Press; New York, 1977; p 49).

To highlight the effectiveness of selective precipitation of contaminating homopolymer from copolymers, polyIA-S was chosen for study because of the contrasting solubility profiles of its constituent homopolymers. Polystyrene swells or dissolves (depending on its molecular weight) in diethyl ether (Et2O) and is insoluble in MeOH. Poly(N-isopropylacrylamide) is insoluble in Et2O but completely miscible with MeOH. After IA was heated at 70° C. with either azobisisobutyronitrile (AIBN) as a control or 200, the polymeric products were precipitated from ether, dried, and heated at 130° C. in S with minimal DMF as a cosolvent. The final reaction mixtures then were dissolved in dichloromethane (DCM) and precipitated into MeOH to remove homopolymers of polyIA.

Subsequently, the collected solids were collected by filtration, dried, dissolved in DCM and precipitated into Et2O to remove polys homopolymer. From the control reaction using AIBN as the initiator, a sticky gel was recovered in 3% yield. This material contained a 1:16 ratio of IA:S residues based on 1H NMR analysis. However, a white solid was obtained in 22% yield from the polymerization with initiator 200, integration of the 1H NMR spectrum suggested a 3:1 ratio of IA:S residues. Although NMR analyses does not discriminate definitively between either a block copolymer structure or a blend of homopolymers, a polymer blend would be expected to yield little solid, if any, using the combination of precipitations described. Thus, the significant yield of polymer derived from 100 supplies strong evidence that the product formed was a block copolymer of IA and S.

It is a well observed phenomenon that in the solid state, block copolymers exhibit interesting morphology due to immiscibility between blocks derived from different monomers (An Introduction to Plastics, Elias, H. -G.; VCH; Weinheim, 1993; p 100). Although immiscible homopolymers can separate into two phases, the polymer chains of block copolymers can only separate from unlike polymer chains to a limited extent because of the covalent coupling between blocks. This microphase separation leads to similar blocks aggregating into domains within the matrix of the other blocks; the resulting domain morphology can be observed by transmission electron microscopy (TEM). Following extensive solid-liquid extractions of the copolymer polyIA-S described above using a Soxhlet apparatus, thin films of this polymer were prepared and examined by TEM. The solid material recovered from the Soxhlet extractor formed transparent solutions in THF and CHCl₃, but a translucent mixture was observed in acetone:MeOH (1:1). Acetone swells or dissolves both homopolymers of polys and polyIA, but MeOH is a selective non-solvent for polys. Amphiphilic block copolymers with a suitable hydrophilic/hydrophobic balance form micelle structures in the presence of selective solvents, and the use of MeOH in our polymer solution may assist microphase separation upon drying to the solid state.

The thin polymer films were cast by dipping copper grids (Li et al. J. Am. Chem. Soc. 1996, 118, 10892) into a polyIA-S solution [1% (w/v) in 1:1 acetone:MeOH], dried, and analyzed by TEM. Blends of homopolymers macrophase separate into large amorphous domains as observed by TEM however, the pattern observed for polyIA-S appeared as an ordered array of microspheres. Their spherical shape was confirmed by rotating the copper disk and observing the resulting TEM image. This observed morphology for polyIA-S is consistent with microphase separation of polys blocks from polyIA blocks in a copolymer.

Finally it should be noted that there is a wide range of molecular weights obtained after the “living” polymerization step (2,300 polyIA-VP to 109,000 polyIA-DS) with no obvious correlation between monomer and molecular weight. The yields from the polymerizations are, as expected, highest for homopolymer synthesis (54-85%). Following the second “living” polymerization step the amount of block copolymer isolated is far more variable. Repeatedly where the “living” polymerization utilizes the VP monomer with any homopolymer, the observed yield of copolymer is very low (5-17%) suggesting that “living” polymerization with the VP monomer is particularly inefficient.

Parallel Graft Copolymer Synthesis Utilizing Initiator 4:

Synthesis of the graft copolymers began by simply heating AIBN with 4 and a subset of three vinyl monomers S, DB and VP at 70° C. to generate linear statistical copolymers of class 13 [polyS(4), polyDS(4) and polyVP(4)] containing pendant TEMPO groups. These copolymers were then polymerized at 130 ° C. with S, DS, and VP.

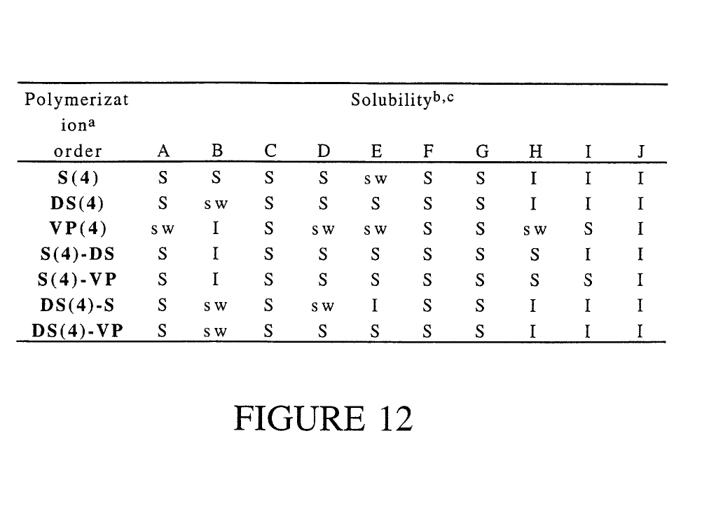

Heating of the copolymer polyVP(4) at 130° C. with either S or DS produced gelatinous reaction mixtures that were insoluble in any solvents listed (table shown in FIG. 12). Gels formed after heating for only 3 min, in contrast to the synthesis of the other comb polymers which were viscous solutions after heating overnight. Hyperbranched polymers have been synthesized previously using a monomer similar in structure to 4 and no observation of any insoluble or crosslinked material was made, although VP monomer was not used.

No gelation was observed when polyVP was mixed with polyS(4) and heated with either S or DS. These results suggest that gel formation is dependent upon the statistical copolymer polyVP(4). In fact, polyVP(4) differed from polyS(4) and polyDS(4) in that its SEC analysis exhibited a bimodal distribution. The exact basis for gelation in this system is speculative, but we postulate that side reactions during polymerization at 130° C. may be leading to crosslinking.

Combinations of S, DS, and VP produced soluble comb polymers with interesting profiles (FIG. 12). Both solubility properties and molecular weights changed considerably after the second polymerization. In contrast to that observed for the block copolymers, the solubility profiles of the comb polymers were generally determined by the second monomer, although not exclusively. For example, both polyS(4)-VP and polyDS(4)-VP are soluble in MeOH although polyS(4) and polyDS(4) are not. However, the presence of polyS(4) and polyDS(4) confers water insolubility on these comb polymers since the homopolymer polyVP is highly soluble in water.

Solubility profiles of block and graft copolymers derived from the same pair of monomers exhibited minor differences (FIGS. 10 and 12). For example, the block copolymer polyDS-S was found to be soluble in both acetone and acetonitrile, whereas polyDS(4)-S only swelled or remained undissolved. Such variances might originate from the acrylate structure derived from 4 in addition to differences in polymer composition and molecular weight. Whatever their source of diversity, these comb polymers provide additional versatility as supports for soluble polymer organic synthesis while exploiting the same monomer set as before.

Selection and Utility of a Block Copolymer for LPOS: After screening the solubility profiles of the individual members of the copolymer library (FIG. 10) polyBS-DS was selected for further study. Of critical importance is its solubility in Et2O and THF and its insolubility in H₂O. This is in complete contrast to the present soluble polymer of choice in LPOS, poly(ethylene) glycol (PEG). The poor solubility of PEG in Et2O and THF has meant that the breadth of chemistry that can be achieved with this support is ultimately limited. An additional problem with PEG-supported chemistry is the polymer′s high solubility in H₂₀ meaning that aqueous extractions to remove salts cannot easily be performed.

Therefore polyBS-DS seemed an ideal starting point for the characterization of a new soluble support for LPOS. As described vide supra block copolymers derived from bifunctional initiators 100, 200 and 3 contain an α-nitrile group at the linkage between the blocks. Reduction of these α-nitrile groups yields amines that can serve as loci for polymer-supported organic chemistry. Reaction of the copolymer polyBS-DS with LiAlH₄ in refluxing THF for 2 h gave the amino functionalized polyBS-DS-NH_{2 22} (FIG. 12; For reduction of polymeric nitrites in the presence of ester linkages derived from initiator 100, catalytic hydrogenation using PtO₂ in dioxane/CHCl₃ has been shown to be successful).

Quantitative ninhydrin analysis revealed a loading of 0.14 mmol g-l of amine which, based on the SEC determined value of Mn =17,000, approximates to 2 amino groups per polymer chain as expected. This value compares favorably with the maximal loading capacity of 0.20 mmol g-1 calculated for PEG monomethyl ether (Mn 5000).

Kinetic Analysis of Imine Formation with polyBS-DS-NH_{2 22:}

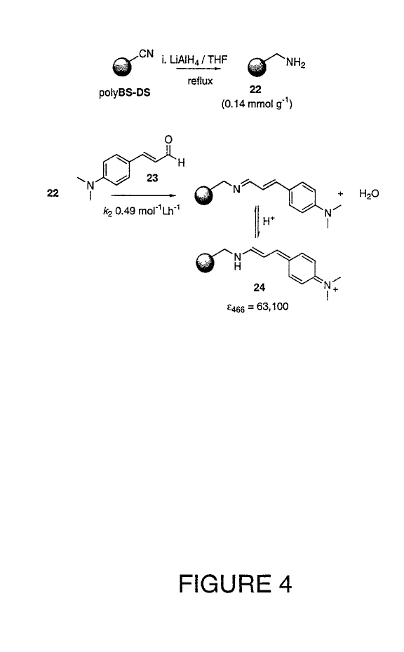

While the functional basis of LPOS is that molecules which are bound to soluble polymer supports often exhibit similar reactivity as their unbound counterparts (Bayer et al. J. Am. Chem. Soc. 1974, 96, 5614; Bayer et al. Peptides 1974; John Wiley & Sons; New York, 1975; p 129; Mutter et al. Int. J. Peptide Protein Res. 1979, 13, 274; Mutter et al. in The Peptides, Vol. 2; Academic; New York, 1979; p 285) it was important to determine that this was the case for our new support poly22. Given that the location of the amino groups of poly22 is in the middle of the block copolymer it was a concern that either one of the polymer blocks may impede their availability for reaction. A comparative kinetic analysis was performed between poly22 and 1-aminohexane for their reaction with 4-dimethylaminocinnamaldehyde 23 (FIG. 4). The rate of iminium ion 24 formation was determined by the method of initial rates in a UV assay by repetitive scanning of a CHCl₃ solution at 466 nm ( Gargiulo et al. J. Am. Chem. Soc. 1994, 116, 3760).

The second order rate constants for imine formation were measured as kpoly22=0.49 L mol-1 h-1 and kaminohexane=0.69 L mol-1 h-1 suggesting that the amino groups of poly22 are indeed sufficiently solvent exposed to make them amenable for derivatization and hence that polyBS-DS is a suitable support for LPOS.

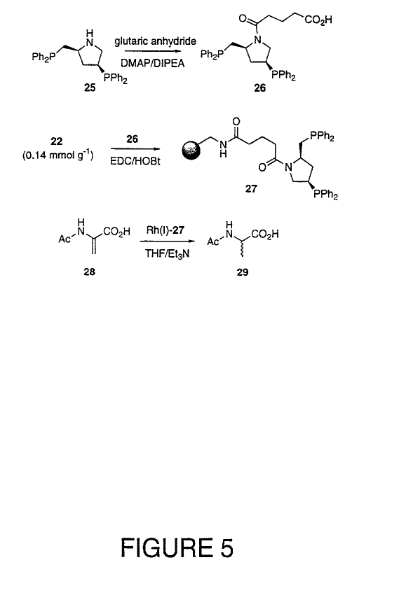

For an application of polyBS-DS in a different setting, we studied its utility as a ligand support in a well characterized rhodium(I) catalyzed asymmetric hydrogenation (FIG. 5; For leading papers on polymer-supported transition metal-catalyzed reactions, see Masuda et al. J. Am. Chem. Soc. 1978, 100, 268; Baker et al. J. Org. Chem. 1981, 46, 2954).

The commercially available diphosphine ligand 25 was treated with glutaric anhydride to generate the glutaroyl derivative 26. An excess of 26 (5 eq.) was then reacted with polyBS-DS-NH_{2 22} (Mn 17,000, 0.14 mmolg-1m) under EDC/HOBt coupling conditions in DCM. The reaction was followed by quantitative ninhydrin analysis, which showed the reaction to be complete after 4 h. The work-up involved simple dropwise addition of the reaction mixture to cold, anhydrous MeOH. The diphosphine derivatized polymer 27 then was collected by filtration, washed repeatedly with MeOH and dried in vacuo. The reaction of 27 with rhodium(I) in the form of [Rh(1,5-cyclooctadiene)Cl]2 in THF gave a light yellow polymer of a Rh(I)-27 complex after isolation by filtration following precipitation into cold, anhydrous MeOH. The reduction of 2-N-acetamidoacrylic acid 28 to 2-N-acetylalanine 29 was performed at 20psi H₂ and 20 ° C. in THF, with a rhodium/phosphine ratio of 0.5 and a substrate/rhodium ratio of 50. As described previously the excess of phosphine ensures that any phosphine sites that had been oxidized during complex formation would not coordinate to rhodium.42 Rh(I)-27 catalyzed the reduction of 28 with a rate comparable to that of the unbound ligand (2S, 4S)-1-tert-butoxycarbonyl -4-diphenylphosphino-2-(diphenylphosphinomethyl)pyrrolidine, 50% vs 40% after 2.5 d respectively. The enantiomeric excess (ee) was determined by an HPLC assay following acylation of the products with an excess of (R)-(+)-1-(1-naphthyl)ethylamine.

The Rh(I)-27 support gave a comparable ee (S-29, 87% ee) to that of the solution based phosphine ligand (S-29, 88% ee). The use of the Rh(I)-27 polymer support had the advantages that precipitation of the polymer-bound ligand with methanol simplified the reaction work up and allowed near quantitative recovery of the expensive ligand essentially unchanged such that recycling was possible.

Polymer Supports with Functionality Derived from TEMPO: As discussed vide supra “living” free radical polymerization with initiators 100, 200, 3-4 may produce polymer chains terminated by TEMPO that, in principle, may be removed by hydrolysis to reveal hydroxyl moieties suitable as orthogonal tether points for functionalization. It was envisioned primarily that the orthogonal nature of loci unmasked from the α-nitrile and TEMPO groups may serve in combinatorial library construction for example, where tagging and/or encoding strategies are required alongside the library development.

Treatment with Zn/AcOH or Zn/NH₄Cl is a well characterized process for cleavage of the N—O bond of TEMPO in small molecules (Barrett et al. J. Org. Chem. 1990, 55, 5196; Shishido et al. J. Org. Chem. 1992, 57, 2876; Boger et al. J. Org. Chem. 1995, 60, 1271). However, in our hands this method gave inconsistent results for the library of block copolymers described. Of preliminary concern was the insolubility of a number of the library members in the Zn/AcOH reaction mixture. Attempts to solubilize these copolymers by addition of cosolvents lowered the yield of TEMPO deprotection in control reactions. Other reductive methods employing Ra-Ni/H₂O, Pd-C/H₂, and SmI2 reportedly failed to cleave the N—O bond of TEMPO, and in fact we have found that using freshly activated zinc, NiCl₂-LiAlH₄, or MO(CO)646 also did not give satisfactory results.

To be able to reproducibly derivatize end groups based on the known termination mechanism of TEMPO in the “living” free radical mechanism of polymerization we have developed initiator 3 (see FIG. 1). The TEMPO groups are themselves now functionalized with Boc protected amino groups. Sequential polymerization of monomers BS followed by DS with initiator 3 produced the soluble support polyBS-DS-(NBoc) (Mn 20,400), a homologue of polyBS-DS described vide supra. A second support, polyVP-S-(NBoc) (Mn 52,200), was formed from tandem polymerization of VP and S. The ease of Boc deprotection was studied for both of these polymer supports in a TFA/DCM (1:10) mixture. Quantitative ninhydrin analysis revealed that the deprotection was complete after stirring overnight. Loadings were measured as 0.06 mmol g-1 (1.3 amino groups per polymer chain) and 0.01 mmol g-1 (0.5 amino groups per polymer chain), respectively. No cleavage of the ester linkages was detected by SEC during this deprotection strategy.

Thus, soluble supports derived from initiator 3 may contain up to four uniformly distributed amino groups after reaction with both H_{2/}PtO₂₄₀ and TFA.

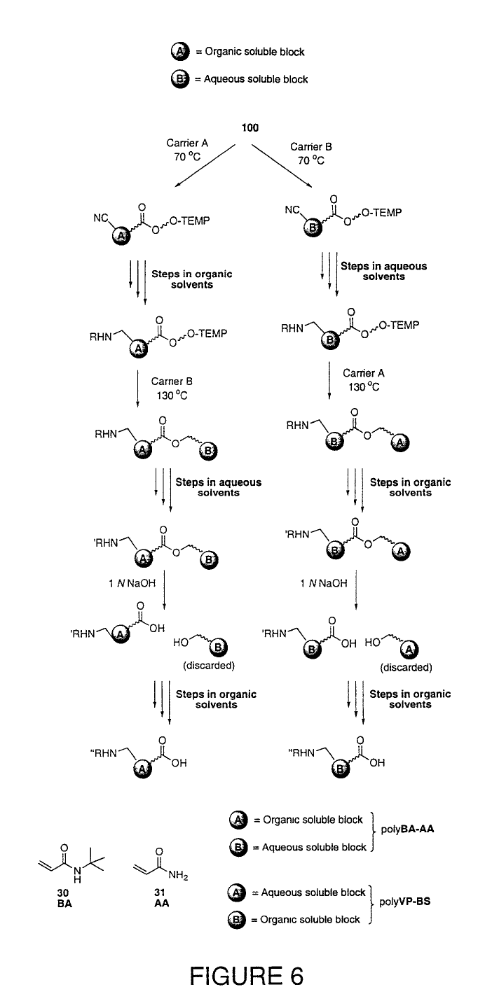

Oscillating Liquid-Phase (OLP) Supports: Bifunctional initiators provide for two independent rounds of polymerization to produce block copolymers in a temperature controlled manner. After the first polymerization, the solubility properties of the newly formed polymer support can be altered considerably by the second round of polymerization which provides the block copolymer support with solubility properties intermediary between the two homopolymers. It is this two-dimensional polymerization approach that allows access to a concept we have dubbed “oscillating liquid-phase” (OLP) synthesis (FIGS. 2 and 6). In this OLP strategy, it is envisioned that molecules can be attached to the homopolymer created by heating bifunctional initiator 100 at 70° C. with a selected monomer (either organic soluble or aqueous soluble). After performing reactions with the homopolymer-bound substrate, the solubility properties of the polymer support then can be changed by the second polymerization (at 130° C.) with a monomer of opposite solubility properties.

Finally, if required the ester linkage between the copolymer blocks may be cleaved during a synthesis to reduce the support to its original solubility as a homopolymer. Thus solubilities can change from organic to aqueous and then back to organic, or vice versa and therefore may be of considerable use in chemistries that require a combination of organic and bioorganic syntheses (Elmore et al. J. Chem. Soc. Chem. Commun. 1992, 1033; Schuster et al. J. Am. Chem. Soc. 1994, 116, 1135; Halcomb et al. J. Am. Chem. Soc. 1994, 116, 11315; Kopper et al. Carbohydr. Res. 1994, 265, 161; Waldmann et al. Angew. Chem. 1997, 109, 642; Yamada et al. Tetrahedron Lett. 1995, 36, 9493; Sauerbrei et al. Angew. Chem. 1998, 110, 1187).

To demonstrate the feasibility of OLP, poly(N-tert -butylacrylamide) (polyBA) was synthesized from initiator 2 (containing an ester linkage) and N-tert-butylacrylamide 30. PolyBA is completely insoluble in water however, a second polymerization with AA yielded a polymer support, polyBA-AA, that forms a translucent aqueous solution. Following treatment with aqueous NaOH, the homopolymer, polyBA was recovered by extraction with ethyl acetate. Cleavage of ester linkages under non-aqueous conditions can also be performed using a methanolic solution of KCN in THF. Similarly, the homopolymer polyVP derived from initiator 100 is a water-soluble support that swells in THF. A second polymerization with BS greatly increases the THF solubility of the block copolymer polyVP-BS.

The transformed copolymer support was also water insoluble, therefore reactions on this new support can involve work-ups that involve aqueous extractions to remove water soluble impurities. These results suggest that it is possible to conduct reactions first in organic solvents (after the first polymerization), then aqueous mixtures (after a second polymerization with a water-soluble monomer), and finally back into organic solutions (after cleavage of the ester linkages between blocks).

### EXAMPLE 2

### Bifunctional Initiators for Free Radical Polymerization of Non-crosslinked block copolymers

We have published a number of soluble polymer supports for the use in alternative methods to solid-phase synthesis (Han et al. Proc. Natl. Acad. Sci. USA 1995, 92, 6419-6423; Han et al. J. Am. Chem. Soc. 1996, 118, 2539-2544; Han et al. J. Am. Chem. Soc. 1996, 118, 7632-7633; Jung et al. Tetrahedron 1997, 53, 6645-6652; Wentworth et al. Chem Commun, 1997, 759-760; Chen et al. J. Am. Chem. Soc. 1997, 119, 8724-8725; Han et al. Angew. Chem. Int. Ed. Engi. 1997, 36, 1731-1733; (h) Gravert, D. J.; Janda, K. D. Chem. Rev. 1997, 97, 489-509). These methods of liquid-phase synthesis (LPS) employ non-crosslinked polymer supports, such as polyethylene glycol (PEG), that completely dissolve in the reaction medium. However, insolubility in both ether and THF (below room temperature) limit the use of PEG in reactions requiring these solvents. Also, the removal of organometallic reagents and inorganic materials during product isolation is complicated by the solubility of PEG in water. Therefore to accommodate a greater variety of reaction conditions, we have developed non-crosslinked block copolymer supports through the use of bifunctional initiators. The prior art, describing block copolymerization using a sequential normal/living radical polymerization scheme, has prompted us to improve and develop new initiators and supports in this area (Li et al. Macromolecules 1997, 30, 5195-5199; Coca et al. Macromolecules 1997, 30, 2808-2810; Coca et al. pi Macromolecules 1997, 30, 6513-6516; Nakagawa et al. Polym. Prepr. Am. Chem. Soc. Div. Polym. Chem. 1997, 38, 701-702; Hawker et al. Macromol. Chem. Phys. 1997, 198, 155-166; Grubbs et al. J. Angew. Chem. Int. Ed. Engl. 1997, 36, 270-272).

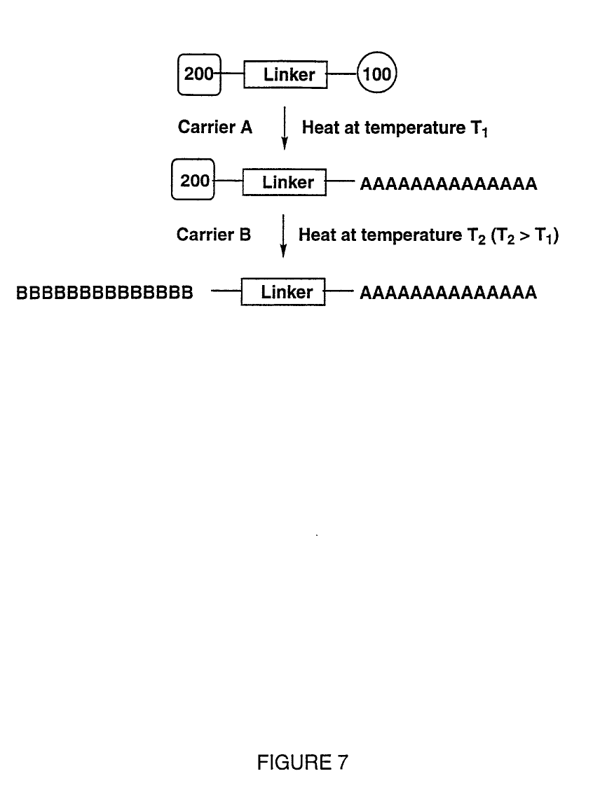

By choosing different monomers and changing block lengths, we envisioned that block copolymers could serve as soluble polymer supports with solubility properties tailored to the designer′s needs. Block copolymers are typically made by anionic polymerization; however, a greater number of vinyl monomers are amenable to radical polymerization (Odian et al. Principles of Polymerization; 2nd Ed.; John Wiley & Sons, Inc.: New York, 1981; pp 181-195). Therefore to provide for a diverse set of block copolymers, bifunctional initiators were synthesized with functional groups that independently produce free radicals at two different temperatures. Block copolymers then could be synthesized by heating the initiator and monomer at one temperature, isolating the resulting polymer, and finally heating the polymer with a different monomer at a higher temperature (FIG. 7). The ability to isolate the intermediate polymer allows the chemist to determine the solubility properties of the polymer support and “fine tune” macromolecular characteristics through a second polymerization.

Thus, bifunctional initiators 100 and 200 were synthesized with both diazene (—N═N—; reference: Moad et al. The Chemistry of Free Radical Polymerization; Pergamon Press: Oxford, 1995; pp. 53-65) and 2,2,6,6-tetra-methyl-piperidinyl-1-oxy (TEMPO) moieties tethered by an ester or ether linkage to produce soluble polymer supports of different chemical stability (Rizzardo et al. Chem. Aust. 1987, 32; Georges et al. Macromolecules 1993, 26, 2987-2988; Hawker et al. J. Am. Chem. Soc. 1994, 116, 11185-11186; Fukuda et al. Macromolecules 1996, 29, 3050-3052; Listigovers et al. Macromolecules 1996, 29, 8992-8993; Hawker et al. Macromolecules 1995, 29, 2993-2995; Hawker et al. , C. J. Angew. Chem. Int. Ed. Engl. 1995, 34, 1456-1459).

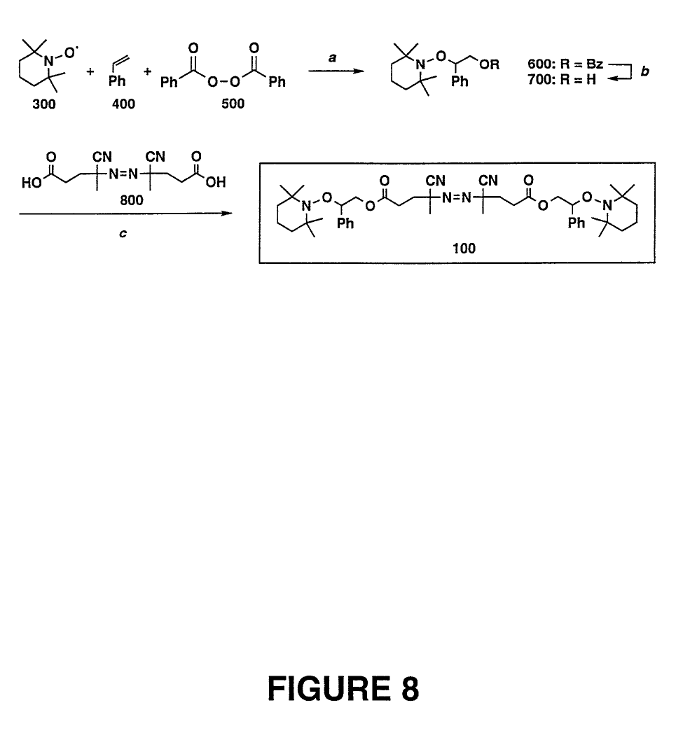

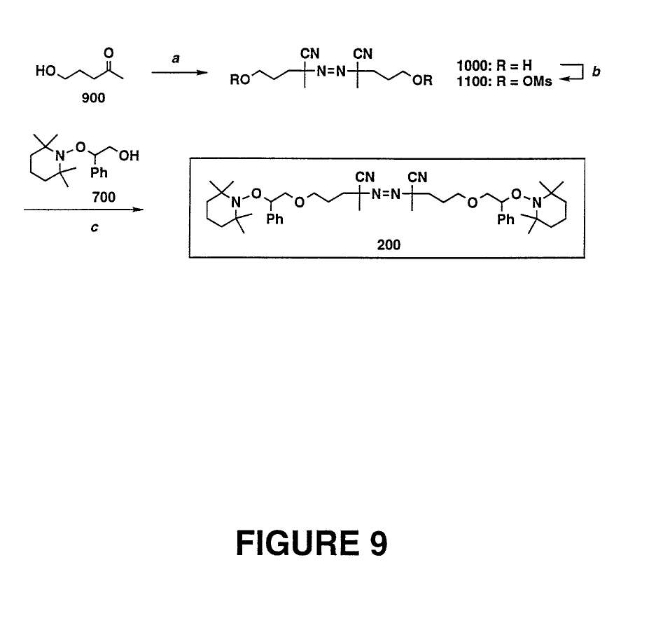

As shown in FIGS. 8 and 9, the convergent syntheses of both 100 and 200 share a common TEMPO-containing intermediate (700) that was prepared from a modified literature procedure (Hawker et al. Macromolecules 1995, 29, 2993-2995). Benzoyl peroxide (110 g; CAUTION: violently decomposes when heated) was added in portions to a degassed solution of 2,2,6,6-tetra -methyl-piperidinyl-1-oxy (TEMPO, 50 g) in styrene (525 mL). The temperature of the exothermic reaction was held between 20-30° C. by frequent application of an ice bath (The heat of solution released upon dissolving the large amount of BPO in styrene certainly contributed to the observed exotherm.

Using a thermometer submerged in the reaction mixture, it was observed that BPO precipitated from solution if allowed to cool below 20° C. When the chilled solution was removed from the ice bath, the reaction temperature soon approached 30° C., and the solution was cooled again) and when the exotherm subsided (2 hr), the reaction was heated at 50° C. overnight. Cooling the mixture precipitated benzoic acid/benzoyl peroxide which was filtered, pressed, and discarded. The filtrate was concentrated by rotary evaporation and purified by chromatography on silica gel (24:1 hexanes:ethyl acetate). The resulting yellow oil was diluted with an equal volume of methanol and stored in a refrigerator overnight to obtain 600 as a white crystalline solid. The requisite intermediate 700 was obtained in pure form without chromatography through room temperature hydrolysis of 600 using 3:1:1 THF:methanol:10 N NaOH. Based on the most expensive reagent (TEMPO), the overall yield for the synthesis of 700 was twice that previously reported (34% vs. 16.5%; Hawker et al. Macromolecules 1995, 29, 2993-2995).

The synthesis of 100 was completed by 1-(3-dimethyl -aminopropyl)-3-ethyl-carbodiimide (EDC)/hydroxybenzotriazole (HOBT) mediated coupling of 700 with commercially available diazene 800 (FIG. 8). Thus, 100 was obtained in one less step with a higher coupling yield (65%) than the reported procedure (Li et al. Macromolecules 1997, 30, 5195-5199) using the acid chloride of 800 (28% yield; Selected analytical data for 100: Rf=0.25 [silica gel, hexanes:diethyl ether:ethyl acetate (6:1:1m)]; 1H NMR (400 MHz, CDCl₃): δ=7.33-7.22 (m, 10H, ArH), 4.93 (t, 2H, J=7.9 Hz, PhCH), 4.62 (m, 2H, PhCHCHHO), 4.32 (ABq, 2H, J=15.8 Hz, PhCHCHHO), 2.27 (m, 4H, CH₂CO₂), 1.60, 1.50, 1.38, 1.32, 1.18, 1.03, 0.68 (each br s, 46H, 8×CH₂ and 10×CH₃); ¹³C NMR (CDCl₃): δ=17.1, 20.3, 23.7, 29.0, 32.9, 33.9, 40.3, 60.0, 66.4, 71.7, 83.7, 117.3, 127.5, 127.8, 128.2, 140.2, 170.8; HRMS: calcd for C₄₆H₆₆N₆O₆ (M+Cs+) 931.4098, found 931.4115.

Soluble polymer supports derived from 100 will contain an ester linkage that may be labile under certain reaction conditions. A copolymer incorporating ester bonds between polymer blocks may be suitable for some applications in materials science (Li et al. Macromolecules 1997, 30, 5195-5199); however, we require polymer supports that can withstand a variety of reaction conditions including the use of reagents that might attack the labile ester linkage. Therefore, the need for block copolymers containing the more stable ether linkage motivated the synthesis of 200 (FIG. 9). Although not commercially available, the diazene 100 was synthesized by a one-pot literature procedure (Bamford et al. Trans.-Faraday Soc. 1960, 56, 932-942). After the mesylate 1100 was obtained by standard methods, etherification was successfully performed using KH in DMSO/THF to provide 200 (Selected analytical data for 200: Rf=0.05 [silica gel, hexane:diethyl ether:chloroform (8:1:1)]; ¹H NMR

(400 MHz, CDCl₃): δ=7.33-7.22 (m, 10H, ArH), 4.80 (t, 2H, J=7.6 Hz, PhCH), 3.92 (m, 2H, PhCHCHHO), 3.58 (m, 2H, PhCHCHHO), 3.40 (m, 4H, OCH₂CH₂), 1.86 (m, 4H, OCH₂CH₂), 1.61, 1.49, 1.36, 1.19, 1.03, 0.64 (each br s, 46H, 8×CH₂ and 10×CH₃); ¹³C NMR (CDCl₃): d=17.2, 20.4, 23.8, 24.5, 33.8, 35.0, 40.5, 59.7, 60.2, 69.8, 72.4, 73.3, 85.2, 85.4, 118.2, 127.3, 127.8, 128.5, 141.9; HRMS: calcd for C₄₆H₇₀N₆O₄ (M+Cs+) 903.4513, found 903.4547) as a pale yellow oil in 64% yield after purification by column chromatography.

Surprisingly, no reaction occurred in THF alone (Hawker has synthesized ethers from 700 using NaH in refluxing THF (references as above); however, the final coupling reaction to 200 cannot be heated due to the thermal lability of diazene (100). The bifunctionality of 100 and 200 provides for two independent rounds of polymerization to produce block copolymers in a temperature controlled manner. Preliminary results include the synthesis of several block copolymers by heating 100 or 200 with one monomer at 70° C., isolating and characterizing the resulting polymer, and incubating the TEMPO -containing macromolecule with a different monomer at 130° C. In fact, from a set of 5 different monomers, polymerizations were conducted in parallel using 200 and various monomer combinations in order to synthesize a two-dimensional array of block copolymers. The non-crosslinked soluble supports display a wide range of solubility profiles, and studies are underway to utilize these novel polymer supports in liquid-phase synthesis.

While a preferred form of the invention has been shown in the drawings and described, since variations in the preferred form will be apparent to those skilled in the art, the invention should not be construed as limited to the specific form shown and described, but instead is as set forth in the following claims.

### EXPERIMENTAL PROTOCOLS

General: Unless otherwise stated, all reactions were performed under an inert atmosphere with dry reagents and solvents and flame-dried glassware. Analytical thin-layer chromatography (TLC) was performed using 0.25 mm coated silica gel Kieselgel 60 F254 plates. Visualization was by UV absorbance, methanolic sulfuric acid, iodine and bromocresol green. 1H NMR spectra were recorded on either a Bruker AMX-500, AMX-400, or AC-250 spectrometer at 500, 400 or 250 MHz respectively. Chemical shifts are reported in parts per million (ppm) on the d scale from an internal standard. 13C NMR spectra were recorded on either a Bruker AMX 500 spectrometer at 125 MHz or a Bruker AMX 400 spectrometer at 100 MHz. High resolution mass spectra were recorded on a VG ZAB-VSE mass spectrometer. UV-vis spectroscopy was performed on a Hewlett Packard 8452A Diode Array Spectrophotometer. Lower critical solution temperatures (LCST) were measured by observing a droplet of polymer solution set directly on a Koffler hot stage melting point apparatus. Size exclusion chromatography (SEC) was performed on a Hitachi L-6200 Intelligent liquid chromatograph pump equipped with a Hitachi D-2000 integrator and either a Hitachi L-4000 UV-vis detector (254 nm) or Hewlett Packard HP 1047A refractive index detector. THF or CHCl₃ (due to low solubilities of some polymers in THF) was used as the mobile phase with a flow rate of 1 mL/min. Three Styrogel (Waters) columns were run in series (7.8×300 mm; 104 Å, 103 Å, 500 Å) and calibrated with ten monodisperse (Mw/Mn<1.13) polystyrene standards obtained from Polymer Laboratories (Mn: 3.15×106, 1.29×106, 6.295×105, 1.706×105, 6.60×104, 2.85×104, 1.29×104, 5.46×103, 1.70×103, 580).

Transmission Electron Microscopy (TEM) was performed with a Philips CM100 electron microscope at 80 kV and data documented on Kodak S0163 film. Polymer films were prepared for imaging by dipping copper mesh grids (3 mm diameter, 200 mesh) into a 1% (w/v) polymer solution (1:1 acetone:methanol) and dried at ambient pressure (overnight) and under vacuum (4 h).

1-Hydroxy-2-phenyl-2-(2′2′, 6, 6′- tetra-methyl-1-piperidinyloxy)ethane and the bifunctional initiators 100 and 200 were prepared using the following conditions, and the standard work-up and stepwise synthetic protocols as described below for the interemediates found in FIGS. 1-6. These conditions were used to synthesize 100 and 200: For 100: step 1: 1 equiv. 3, 15 equiv. 4, 3 equiv. 5, <30° C., 2 h, then 50° C., 16 h, 38%; step 2: 12 equiv. NaOH, THF/MeOH/water (3:1:1), 20° C., 16 h, 89%; step 3: 2.05 equiv. 7, 1 equiv. 8, 3.6 equiv. EDC, 3.6 equiv. HOBT, DMF, 20° C., 72 h, 65%. For 200: step 1: 1.9 equiv. 9, 1 equiv. hydrazine sulfate, 2 equiv. NaCN, water, 20° C., 72 h, then excess conc. HCl followed by 1 equiv. Br2 over 4 h, 0° C., 29%; step 2: 3 equiv. MsCl, 3 equiv. NEt3, CH₂Cl₂, 20° C., 2 h, 95%; step 3: 2.1 equiv. 7, 2.06 equiv. KH, 2.1 equiv. DMSO, THF, 10 min, 20° C., then transferred to 11 in THF, 90 min, 20° C., 64%. Reversed phase HPLC was performed on a Hitachi LC6000 series machine with an Adsorbosphere HS RP-C18 analytical column.

Synthesis of Initiators 3 and 4. 4-(tert-butoxy-carbonyl -amino-2,2,6,6-tetramethyl-1-piperidinyloxyethane 15 as shown in FIG. 3. A solution of 4-amino-(2,2,6,6-tetramethyl-1-pepridinyloxy)ethane (2g, 11.6 mmol), di-tert-butyldicarbonate (3.2 mL, 14 mmol) and diisopropylethylamine (DIPEA, 4.2 mL, 24 mmol) in DCM (100 mL) was stirred for 4 h at rt. The reaction mixture then was diluted with DCM (100 mL) and washed with 1 N HCl (3×250 mL), and brine (2×250 mL). The combined organic fractions were combined, dried (Na2SO₄) and evaporated in vacuo, to give a crude orange oil which was purified by silica gel chromatography (DCM/MeOH 95:5). This gave 15 as a pale orange solid (2.4 g, 76%). 1H NMR (CDCl₃) d 4.4 (bs, 1H). 3.9 (bs, 1H), 2.0 (bd, 1H), 1.56 (s, 9H, tert-butyl), 1.51 (s, 3H, CH₃), 1.49 (bs, 2H), 1.48, (s, 3H, CH₃), 1.27 (s, 3H, CH₃), 1.15 (s, 3H, CH₃) ; 13C NMR (CDCl₃) d 155.45, 80.09, 60.48, 45.99, 31.78, 28.90, 28.14, 27.37; LRESMS+(M+Na)+295.

1-Benzoyloxy-(4-tertbutoxycarbonylamino)-2-phenyl-2-(2,2,6,6-tetramethyl-1-piperidinyloxy)ethane 16 as shown in FIG. 3. A solution of 15 (1.7 g, 6.3 mmol) and benzoylperoxide (1.52 g, 6.3 mmol) in styrene 5 (50 mL) was stirred at 50° C. overnight. Following evaporation of the volatiles in vacuo the residue was purified by silica gel chromatography (DCM/MeOH 98:2). This gave 16 as a fluffy white solid (1.2 g, 38%). 1H NMR (CDCl₃) d 7.8 (d, 2H, Ar-H), 7.4 (t, 1H, Ar-H), 7.30-7.05 (complex m, 7H, Ar-H), 4.95 (t, 1H, CH), 4.80 (dd, 1H, CH), 4.3 (dd, 1H, CH), 4.2 (bs, 1H), 3.8 (bs, 1H), 1.94-1.72 (complex m, 2H), 1.60 (s, 9H, tert-butyl), 1.51 (s, 3H, CH₃), 1.48, (s, 3H, CH₃), 1.27 (s, 3H, CH₃), 1.15 (s, 3H, CH₃); 13C NMR (CDCl₃) d 166.31, 155.21, 140.29, 133.41, 132.89, 130.08, 129.66, 128.39, 128.33, 128.23, 84.07, 79.31, 66.61, 60.45, 46.55, 42.03, 33.91, 28.40, 20.93 ; HRFABMS calcd for C₂₉H₄₁N205 497.3015; obsd 497.3002.

1-Hydroxy-2-phenyl-2-(4-tertbutoxycarbonylamino-2,2,6,6-tetramethyl-1-piperidinyloxy)ethane 17 as shown in FIG. 3. The ester 16 (0.9 g, 1.8 mmol) was dissolved in a 10 N NaOH/THF/MeOH (3:1:1) mixture (16 mL) and stirred for 8 h at rt. The reaction mixture was then diluted with diethyl ether (50 mL) and partitioned. The organic fraction was washed with brine (2×50 mL), dried (Na2SO₄) and evaporated in vacuo . The crude residue was purified by silica gel chromatography (DCM/MeOH 95:5) to give alcohol 17 as a white solid (700 mg, 98 %). ¹H NMR (CDCl₃) d 7.25 (bm, 5H, Ar-H), 5.2 (dd, 1H, CH), 4.35 (bs, 1H), 4.2 (dd, 1H, CH), 3.87 (bs, 1H), 3.75 (dd, 1H, CH), 1.95-1.85 (complex m, 2H), 1.65 (s, 3H, CH₃), 1.60 (s. 9H. tert-butyl), 1.35-1.00 (complex m, 1lH, 3×CH₃ and CH₂); 13C NMR (CDCl₃) d 155.19, 139.92, 128.21, 127.93, 127.67, 83.89, 79.28, 66.62, 60.48, 46.52, 32.97, 28.40, 20.90. HRFABMS calcd for C₂₂H₃₆N204 393.3675; obsd 393.3672.

Diazene 3 as shown in FIG. 3. A solution of alcohol 17 (700 mg, 1.78 mmol), the diacid 18 (180 mg, 0.71 mmol), EDC (544 mg, 2.84 mmol), hydroxybenzotriazole (HOBt, 383 mg, 2.84 mmol) and DIPEA (0.74 mL, 4.26 mmol) in THF (10 mL) was stirred for 8 h at rt. The reaction mixture was then diluted with diethyl ether (50 mL) and washed sequentially with 1N HCl (3×50 mL), saturated NaHCO3 (3×50 mL) and brine (2×50 mL). The combined organic fractions were then combined, dried (Na2SO₄) and evaporated in vacuo to give a crude colorless oil which was purified by silica gel chromatography (Et2O/hexane 1:1). This gave initiator 3 as a white crystalline solid (557 mg, 76%). 1H NMR (CDCl₃) d 7.34-7.2 (complex m, 10H, Ar-H), 4.89 (dd, 2H, CH), 4.60 (m, 2H, CH), 4.31-4.24 (complex m, 3H), 3.75 (bs, 1H), 2.39-2.17 (complex m, 8H, 4×CH₂), 1.79 (d, 2H), 1.67 (d, 2H), 1.60 (s, 3H, CH₃), 1.58 (s, 3H, CH₃), 1.42 (s, 18H, 2×tert -butyl), 1.42 (s, 6H, 2×CH₃), 1.32 (s, 6H, 2×CH₃), 1.30-1.28 (m, 2H), 1.16 (s, 6H, 2×CH₃), 0.65 (s, 6H, 2×CH₃); 13C NMR (CDCl₃) d 170.84, 155.19, 139.92, 129.02, 127.93, 127.67, 117.41, 83.89, 79.28, 71.76, 66.40, 60.48, 46.52, 41.99, 33.84, 32.97, 30.29, 28.96, 23.81, 23.47, 20.90; HRFABMS calcd for C₅₆H₈₄N8O₁₀Cs 1161.5365; obsd 1161.5437.

1-Methacryloyloxy-2-phenyl-2-(2,2,6,6′-tetramethyl-1-piperidinyloxy)ethane 4 as shown in FIG. 3. 1-Hydroxy-2-phenyl-2-(2,2,6,6′-tetramethyl-1-piperidinyloxy)ethane 19 (15 g, 54 mmol, 1 equiv) was dissolved in dry DCM (150 mL). Triethylamine (12.2 mL, 87.5 mmol, 1.6 equiv) was added followed by methacryloyl chloride 20 (7.9 mL, 82 mmol, 1.5 equiv), and an ice bath was applied briefly. After stirring under a nitrogen atmosphere for 2.5 h, the reaction mixture was washed with 1 N HCl (3×100 mL), brine (100 mL), dried (Na2SO₄), and evaporated to dryness. The crude product was purified by column chromatography (19:1 hexane:ethyl acetate). This gave 4 as a white solid (12.1 g, 65% ). 1H NMR (400 MHz, CDCl₃) d 7.33-7.22 (m, 5H, Ar-H), 5.98 (s, 1H, CH₂), 5.47 (s, 1H, CH₂), 4.95 (t, 1H, CH), 4.62 (dd, 1H, CH), 4.33 (dd, 1H, OH), 1.84 (s, 3H, CH₃), 1.62, 1.48, 1.37, 1.31, 1.17, 1.03, 0.70 (each br s, 18H, 3×CH₂ and 4×CH₃); 13C NMR (CDCl₃) d 167.14, 140.64, 136.15, 127.93, 127.52, 125.53, 83.83, 66.47, 60.03, 40.37, 33.96, 20.28, 18.26, 17.10. HRFABMS: calcd for C₂₁H₃₁NO₃ (M+Na)+368.2202; obsd 368.2214.

General Procedure for Block and Graft Copolymer Synthesis. Monomers were distilled prior to use except for acrylamide and its derivatives which were used as received. Polymerization yields were determined gravimetrically and calculated from a theoretical yield based on 100% monomer conversion. Although often too small and/or broad to accurately determine, some initiator-derived resonances were observed by 1H and 13C NMR analysis and are reported in those instances. The general method for copolymer synthesis is illustrated once each for formation of the block copolymer polyS-BS and graft copolymer polyS(4)-DS. For homopolymer synthesis only the first polymerization at 70 oC occurs. Note that the precipitation solvents change for each copolymer.

Blockpolymer synthesis. 1. Homopolymers. Polystyrene (S).

A solution of 300 mg of 200 (300 mg, 0.39 mmol) and styrene 5 (0.89 mL, 7.75 mmol) in DCB (3 mL) was freeze thawed 3 times with liquid nitrogen and then heated at 70° C. for 8 h. The solution was precipitated by dilution in DCM followed by dropwise addition into MeOH to give a white solid, yield 80%. Mn(THF)=8,000 and PD=2.11; ¹H NMR (CDCl₃) d 7.25-6.8 (br m, Ar-H), 6.8-6.2 (br m, Ar-H), 2.1-1.65 (br s, polymer backbone), 1.65-1.2 (br s, polymer backbone); 13C NMR (CDCl₃): d 145.27, 127.83, 125.66, 85.38, 73.35, 70.44, 40.47, 34.05, 20.38, 17.16.

Poly(4-tert-buty1styrene) (BS). Reaction: 300 mg of 200 (0.39 mmol, 1 equiv) and 4-tert-butylstyrene 6 (1.42 mL, 7.75 mmol, 20 equiv) in DCB (3 mL). Precipitation: DCM/methanol to give a white solid, yield 85%. Mn(THF)=24,000 and PD=2.36; 1H NMR (CDCl₃) d 7.35-6.8 (br m, Ar-H), 6.8-6.05 (br m, Ar-H), 2.15-1.5 (br m, polymer backbone), 1.5-1.1 (br s, t-butyl group and polymer backbone); 13C NMR (CDCl₃): d 147.95, 142.72, 127.21, 124.61, 85.33, 40.47, 39.79, 34.23, 31.53, 20.32, 17.16.

Poly(3,4-dimethoxystyrene) (DS). Reaction: 300 mg of 200 (0.39 mmol, 1 equiv) and 3,4-dimethoxystyrene 7 (1.15 mL, 7.75 mmol, 20 equiv) in DCB (3 mL). Precipitation: DCM/methanol to give a white solid, yield 75%. Mn(THF)=19,000 and PD=1.67; 1H NMR (CDCl₃) d 6.75-6.25 (br m, Ar-H), 6.25-5.75 (br m, Ar-H), 3.95-3.4 (br d, -OCH₃), 2.2-1.6 (br s, polymer backbone), 1.6-1.2 (br s, polymer backbone); 13C NMR (CDCl₃): d 148.34, 147.02, 137.94, 127.71, 127.13, 119.42, 110.49, 85.26, 73.26, 70.33, 55.59, 44.72, 40.13, 33.99, 20.25, 17.07.

Poly(N-vinylpyrrolidinone) (VP). Reaction: 140 mg of 200 (0.18 mmol, 1 equiv) and N-vinylpyrrolidinone 8 (0.39 mL, 3.6 mmol, 20 equiv) in DCB (1.5 mL). Precipitation: methanol/diethyl ether, then DCM/hexane to give a white powder, yield 74%. Mn(CHCl₃)=5,100 and PD=1.44; 1H NMR (CDCl₃) d 4.05-3.5 (br m, 1H, CH), 3.5-3.05 (br s, 2H, CH₂), 2.55-1.3 (br m); 13C NMR (CDCl₃) d 175.45, 127.78, 73.27, 44.81, 43.52, 42.34, 31.39, 19.93, 18.26, 18.00.

Poly(N-isopropylacrylamide) (IA). Reaction: 131 mg of 200 (0.17 mmol, 1 equiv) and N-isopropylacrylamide 9 (388 mg, 3.4 mmol, 20 equiv) in DMF (1.5 mL). Precipitation: THF/diethyl ether to give a white solid, yield 54%. Mn(CHCl₃)=18.100 and PD=1.40; 1H NMR (CDCl₃) d 6.8-5.7 (br s, 1H, NH), 4.1-3.9 (br s, 1H, CH), 2.4-1.2 (br m), 1.2-0.95 (br s, 6H, CH₃); 13C NMR (CDCl₃): d 174.54, 127.69, 73.09, 70.76, 42.19, 41.24, 35.10, 22.46, 20.83, 17.45.

2. Copolymers. Polystyrene-Poly(4-tert-buty1styrene) (S -BS) A solution of the homopolymer polys 102 mg (0.98 mmol styrene residues estimated, 1 equiv) in 4-tert-butyl styrene 6 (0.197 mL, 1.08 mmol, 1.1 equiv) was freeze thawed 3 times at −70° C. and then heated at 130° C. for 12 h. The reaction mixture was then diluted with DCM and added dropwise to MeOH. The resultant precipitate was collected by filtration to give polyS-BS as a white powder, yield 44%. Mn(THF)=7,800 and PD -2.42; 1H NMR (CDCl₃) d 7.35-6.05 (br m, Ar-H), 2.15-1.1 (br m, includes tert-butyl group); 1H signal integration: 2.5:1 ratio of styrene:4-tert-buty1styrene residues; 13C NMR (CDCl₃) d 148.18, 145.10, 142.54, 127.31, 125.65, 124.67, 40.38, 34.26, 31.49.

Polystyrene-Poly(3,4-dimethoxystyrene) (S-DS). Reaction: 53 mg polyS (0.51 mmol styrene residues estimated, 1 equiv) and 3,4-dimethoxystyrene 7 (0.114 mL, 0.77 mmol, 1.5 equiv). Precipitation: DCM/methanol to give a white powder, yield 33%. Mn(THF)=8,500 and PD=2.00; 1H NMR (CDCl₃): d=7.25-5.75 (br m, Ar-H), 3.95-3.4 (br d, —OCH₃), 2.2-1.2 (br m); ¹H signal integration: 1.9:1 ratio of styrene:3,4-dimethoxystyrene residues; 13C NMR (CDCl₃) d 148.41, 146.95, 145.27, 136.48, 127.96, 125.65, 119.46, 110.62, 55.67, 40.37.

Polystyrene-Poly(N-vinylpyrrolidinone) (S-VP). Reaction: 42 mg polys (0.40 mmol styrene residues estimated, 1 equiv) dissolved first in 0.07 mL DMF, N-vinylpyrrolidinone 8 (0.344 mL, 3.2 mmol, 8 equiv); heated for 40 h. Precipitation: THF/methanol to give a white powder, yield 5%. Mn(THF)=8,400 and PD=1.75; 1H NMR (CDCl₃): d 7.25-6.8 (br m, Ar-H), 6.8-6.2 (br m, Ar-H), 4.05-3.5 (br m, CH), 3.5-3.05 (br s, CH₂), 2.55-1.2 (br m); 1H signal integration: 3.2:1 ratio of styrene:N -vinylpyrrolidinone residues; 13C NMR (CDCl₃) d 176.23, 145.16, 127.66, 125.62, 44.10, 43.48, 42.30, 40.36, 18.11.

Polystyrene-Poly(N-isopropylacrylamide) (S-IA). Reaction: 43 mg polys (0.41 mmol styrene residues estimated, 1 equiv) dissolved first in 0.07 mL DMF, N-isopropylacrylamide 9 (0.284 g, 2.5 mmol, 6 equiv); heated for 40 h. Precipitation: THF/diethyl ether to give to give polyS-IA as a white powder, yield 16%. Mn(THF)=16,200 and PD=1.61; 1H NMR (CDCl₃) d 7.25-6.2 (br m), 4.1-3.9 (br s, NCH), 2.4-0.95 (br m, includes -CH₃); 1H signal integration: 1:2.4 ratio of styrene:N -isopropylacrylamide residues; 13C NMR (CDCl₃) d 174.38, 145.09, 127.84, 125.61, 42.34, 41.35, 40.37, 22.57.

Poly(4-tert-buty1styrene)-Polystyrene (BS-S). Reaction: 51 mg polyBS (0.32 mmol 4-tert-buty1styrene residues estimated, 1 equiv) and styrene 5 (0.109 mL, 0.95 mmol, 3 equiv). Precipitation: DCM/methanol to give polyBS-S as a white solid, yield 35%; Mn(THF)=19,300 and PD=3.07; 1H NMR (CDCl₃): d=7.35-6.05 (br m, Ar-H), 2.15-1.1 (br m, includes t-butyl group); 1H signal integration: 3.5:1 ratio of 4-tert -butylstyrene:styrene residues; 13C NMR (CDCl₃): d=145.23, 148.01, 142.72, 127.51, 125.25, 124.64, 40.32, 39.81, 34.31, 31.55.

Poly(4-tert-butylstyrene)-Poly(3,4-dimethoxystyrene) (BS -DS). Reaction: 100 mg polyBS (0.62 mmol 4-tert-butylstyrene residues estimated, 1 equiv) and 3,4-dimethoxystyrene 7 (0.102 mL, 0.69 mmol, 1.1 equiv). Precipitation: DCM/methanol to give polyBS-DS as a white solid, yield 59%. Mn(THF)=19,500 and PD =2.44; 1H NMR (CDCl₃) d 7.35-5.75 (br m, Ar-H), 3.95-3.4 (br d, —OCH₃), 2.2-1.1 (br m, includes t-butyl group); 1H signal integration: 2.5:1 ratio of 4-tert-butylstyrene:3,4-dimethoxystyrene residues; 13C NMR (CDCl₃) d 148.34, 148.00, 146.79, 142.78, 137.64, 127.21, 124.61, 119.43, 110.59, 55.66, 40.08, 39.83, 34.24, 31.54.

Poly(4-tert-butylstyrene)-Poly(N-vinylpyrrolidinone) (BS -VP). Reaction: 52 mg polyBS (0.32 mmol 4-tert-butylstyrene residues estimated, 1 equiv) dissolved first in 0.085 mL DCB, N-vinylpyrrolidinone 8 (0.174 mL, 1.6 mmol, 5 equiv). Precipitation: DCM/methanol to give polyBS-VP as a white solid, yield 6%. Mn(THF)=20,500 and PD=2.52; 1H NMR (CDCl₃) d 7.35-6.8 (br m, Ar-H), 6.8-6.05 (br m, Ar-H), 4.05-3.5 (br m, —NCH—), 3.5-3.05 (br s, NCH₂), 2.55-1.1 (br m, includes t-butyl group); 1H signal integration: 2.4:1 ratio of 4-tert -butylstyrene:N-vinylpyrrolidinone residues; 13C NMR (CDCl₃) d 175.43, 147.79, 142.60, 127.34, 124.59, 45.09, 43.94, 42.78, 40.04, 34.23, 31.50, 17.93.

Poly(4-tert-butylstyrene)-Poly(N-isopropylacrylamide) (BS-IA). Reaction: 52 mg polyBS (0.32 mmol 4-tert-butylstyrene residues estimated, 1 equiv) and N-isopropylacrylamide 9 (148 mg, 1.3 mmol, 4 equiv). Precipitation: THF/methanol to give polyBS-IA as a white solid, yield 19%. Mn(THF)=27,100 and PD =2.04; 1H NMR (CDCl₃) d 7.35-6.8 (br m, Ar-H), 6.8-6.05 (br m, Ar-H), 4.1-3.9 (br s, —NCH—), 2.4-0.95 (br m); 1H signal integration: 3.1:1 ratio of 4-tert-butylstyrene:N -isopropylacrylamide residues; 13C NMR (CDCl₃) d 175.24, 147.96, 142.78, 127.31, 124.62, 42.29, 41.35, 39.78, 34.25, 31.50, 22.43.

Poly(3,4-dimethoxystyrene)-Polystyrene (DS-S). Reaction: 58 mg polyDS (0.35 mmol 3,4-dimethoxystyrene residues estimated, 1 equiv) and styrene 5 (0.101 mL, 0.88 mmol, 2.5 equiv). Precipitation: DCM/methanol to give polyDS-S as a white solid, yield 20%. Mn(THF)=17,800 and PD=1.73; 1H NMR (CDCl₃) d 7.25-5.75 (br m, Ar-H), 3.95-3.4 (br d, —OCH₃), 2.2-1.2 (br m); 1H signal integration: 3:1 ratio of 3,4-dimethoxystyrene:styrene residues; 13C NMR (CDCl₃): d=148.24, 146.70, 145.23, 137.24, 127.94, 125.63, 119.45, 110.48, 55.64, 40.23.

Poly(3,4-dimethoxystyrene)-Poly(4-tert-butylstyrene) (DS -BS). Reaction: 54 mg polyDS (0.34 mmol 3,4-dimethoxystyrene residues estimated, 1 equiv) dissolved first in 0.09 mL DCB, 4-tert-butylstyrene 6 (0.182 mL, 0.99 mmol, 3 equiv). Precipitation: THF/methanol to give polyDS-BS as a white solid, yield 30%. Mn(THF)=18,900 and PD=1.72; 1H NMR (CDCl₃) d 7.35-5.75 (br m, Ar-H), 3.95-3.4 (br d, —OCH₃), 2.2-1.1 (br m, includes t-butyl group); 1H signal integration: 2.7:1 ratio of 3,4-dimethoxystyrene:4-tert-butylstyrene residues; 13C NMR (CDCl₃) d 142.93, 148.29, 147.95, 146.81, 137.65, 127.16, 124.76, 119.10, 110.49, 55.62, 40.17, 39.85, 34.29, 31.47.

Poly(3,4-dimethoxystyrene)-Poly(N-vinylpyrrolidinone) (DS -VP). Reaction: 48 mg polyDS (0.29 mmol 3,4-dimethoxystyrene residues estimated, 1 equiv) dissolved first in 0.063 mL DMF, N-vinylpyrrolidinone 8 (0.125 mL, 1.2 mmol, 4 equiv). Precipitation: THF/methanol, filtered solid [polyDS, identified by 1H NMR], concentrated filtrate, then precipitated with hexane to give to give polyDS-VP, yield 17%. Mn(THF)=9,800 and PD=2.34; 1H NMR (CDCl₃) d 6.75-6.25 (br m, Ar-H), 6.25-5.75 (br m, Ar-H), 4.05-3.05 (br m), 2.55-1.2 (br m); 1H signal integration: 1.3:1 ratio of 3,4-dimethoxystyrene:N-vinylpyrrolidinone residues; 13C NMR (CDCl₃) d 175.41, 148.28, 146.73, 137.08, 119.30, 110.56, 55.62, 44.73, 43.44, 42.50, 40.20, 31.38, 18.30.

Poly(3,4-dimethoxystyrene)-Poly(N-isopropylacrylamide) (DS -IA). Reaction: 54 mg polyDS (0.33 mmol 3,4-dimethoxystyrene residues estimated, 1 equiv) and N-isopropylacrylamide 9 (381 mg, 3.4 mmol, 10 equiv). Precipitation: DCM/diethyl ether to give polyDS-IA as a white solid, yield 12%. Mn(THF)=17,000 and PD=1.77; 1H NMR (CDCl₃) d 6.75-6.25 (br m, Ar-H), 6.25-5.75 (br m, Ar-H), 4.1-3.4 (br m), 2.4-0.95 (br m); 1H signal integration: 3.5:1 ratio of 3,4-dimethoxystyrene:N -isopropylacrylamide residues; 13C NMR (CDCl₃) d 174.16, 148.29, 146.73, 137.56, 119.55, 110.52, 55.62, 42.47, 41.25, 40.13, 22.62.

Poly(N-vinylpyrrolidinone)-Polystyrene (VP-S). Reaction: 41 mg polyVP (0.37 mmol N-vinylpyrrolidinone residues estimated, 1 equiv) dissolved first in 0.105 mL DMF, styrene 5 (0.211 mL, 1.8 mmol, 5 equiv). Precipitation: DCM/hexane to give polyVP-S as a white solid, yield 26%. Mn(THF) 10,600 and PD 4.10; 1H NMR (CDCl₃) d 7.25-6.8 (br m, Ar-H), 6.8-6.2 (br m, Ar-H), 4.05-3.5 (br m, NCH), 3.5-3.05 (br s, NCH₂), 2.55-1.2 (br m); 1H signal integration: 1.3:1 ratio of N-vinylpyrrolidinone:styrene residues; 13C NMR (CDCl₃) d 175.45, 145.24, 127.92, 125.62, 44.82, 43.63, 42.12, 40.30, 31.58, 18.31.

Poly(N-vinylpyrrolidinone)-Poly(4-tert-butylstyrene) (VP -BS). Reaction: 32 mg polyVP (0.29 mmol N-vinylpyrrolidinone residues estimated, 1 equiv) dissolved first in 0.10 mL DMF, 4-tert-butylstyrene 6 (0.263 mL, 1.4 mmol, 5 equiv); heated for 58 h. Precipitation: THF/methanol to give polyVP-BS as a white solid, yield 25%. Mn(THF)=5,700 and PD=3.21; 1H NMR (CDCl₃) d 7.35-6.8 (br m, Ar-H), 6.8-6.05 (br m, Ar-H), 4.05-3.5 (br m, NCH), 3.5-3.05 (br s, NCH₂), 2.55-1.1 (br m, includes tert-butyl group); 1H signal integration: 1:2.8 ratio of N -vinylpyrrolidinone:4-tert-butylstyrene residues; 13C NMR (CDCl₃) d 175.54, 148.01, 142.76, 127.34, 124.60, 44.87, 43.58, 42.73, 39.74, 34.29, 31.51, 31.28, 18.32.

Poly(N-vinylpyrrolidinone)-Poly(3,4-dimethoxystyrene) (VP -DS). Reaction: 33 mg VP (0.30 mmol N-vinylpyrrolidinone residues estimated, 1 equiv) dissolved first in 0.044 mL DMF, 3,4-dimethoxystyrene 7 (0.088 mL, 0.059 mmol, 2 equiv). Precipitation: THF/diethyl ether, washed with methanol to give polyVP-DS as a white solid, yield 54%. Mn(THF)=47,100 and PD =2.63; 1H NMR (CDCl₃) d 6.75-6.25 (br m, Ar-H), 6.25-5.75 (br m, Ar-H), 4.05-3.05 (br m), 2.55-1.2 (br m); iH signal integration: 1:1.7 ratio of N-vinylpyrrolidinone:3,4-dimethoxystyrene residues; 13C NMR (CDCl₃) d 175.56, 148.34, 146.81, 137.95, 119.40, 110.48, 55.59, 44.75, 43.46, 42.39, 40.17, 31.42, 18.27.

Poly(N-vinylpyrrolidinone)-Poly(N-isopropylacrylamide) (VP -IA). Reaction: 35 mg polyVP (0.31 mmol N-vinylpyrrolidinone residues estimated, 1 equiv) dissolved first in 0.1 mL DMF, N -isopropylacrylamide 8 (0.182 g, 1.6 mmol, 5 equiv). Precipitation: THF/diethyl ether to give polyVP-IA as a white solid, yield 35%. Mn(CHCl₃)=7,100 and PD=1.57; 1H NMR (CDCl₃) d 6.8-5.7 (br s, NH), 4.1-3.5 (br m), 3.5-3.05 (br s, NCH₂), 2.55-0.95 (br m, includes —CH₃); 1H signal integration: 1:2.4 ratio of N-vinylpyrrolidinone:N-isopropylacrylamide residues; 13C NMR (CDCl₃) d 175.48, 174.61, 44.79, 43.66, 42.41, 41.38, 31.46, 22.52, 18.29; LCST(H₂₀)=38° C.

Poly(N-isopropylacrylamide)-Polystyrene (IA-S). Reaction: 27 mg polyIA (0.24 mmol N-isopropylacrylamide residues estimated, 1 equiv) dissolved first in 0.035 mL DMF, styrene 5 (0.19 mL, 1.66 mmol, 7 equiv) heated for 8 h. Precipitation: THF/diethyl ether to give polyIA-S as a white solid, yield 15%. Mn(THF)=49,100 and PD=1.53; 1H NMR (CDCl₃) d 7.25-6.2 (br m), 4.1-3.9 (br s, —NCH—), 2.4-0.95 (br m, includes —CH₃); 1H signal integration: 1.1:1 ratio of N -isopropylacrylamide:styrene residues; 13C NMR (CDCl₃) d 174.55, 145.38, 127.64, 125.65, 42.36, 41.28, 40.33, 22.58.

Poly(N-isopropylacrylamide)-Poly(4-tert-butylstyrene) (IA -BS). Reaction: 24 mg polyIA (0.21 mmol N-isopropylacrylamide residues estimated, 1 equiv) dissolved first in 0.10 mL DMF, 4-tert-butylstyrene 6 (0.269 mL, 1.47 mmol, 7 equiv); heated for 58 h. Precipitation: THF/methanol to give polyIA-BS as a white solid, yield 41%. Mn(THF)=20,400 and PD=5.92; 1H NMR (CDCl₃) d 7.35-6.8 (br m, Ar-H), 6.8-6.05 (br m, Ar-H), 4.1-3.9 (br s, —NCH—), 2.4-0.95 (br m); 1H signal integration: 1.2:2 ratio of N-isopropylacrylamide:4-tert-butylstyrene residues; 13C NMR (CDCl₃) d 174.62, 148.00, 142.76, 127.35, 124.63, 42.18, 41.32, 39.88, 34.26, 31.53, 22.30.

Poly(N-isopropylacrylamide)-Poly(3,4-dimethoxystyrene) (IA-DS). Reaction: 31 mg polyIA (0.27 mmol N-isopropylacrylamide residues estimated, 1 equiv) dissolved first in 0.1 mL DMF, 3,4-dimethoxystyrene 7 (0.2 mL, 1.35 mmol, 5 equiv). Precipitation: THF/methanol yielded a milky solution which was filtered through cotton to remove solid homopolymer [polyDS, identified by 1H NMR]. The filtrate was concentrated in vacuo and added dropwise to hexane. The precipitate was collected by filtration to give polyIA-DS as a white solid, yield 45%. Mn(THF) 109,000 and PD=1.56; 1H NMR (CDCl₃) d 6.75-6.25 (br m, Ar-H), 6.25-5.75 (br m, Ar-H), 4.1-3.4 (br m), 2.4-0.95 (br m); 1H signal integration: 1:1.7 ratio of N-isopropylacrylamide:3,4-dimethoxystyrene residues; 13C NMR (CDCl₃) d 174.55, 148.37, 146.85, 136.45, 119.42, 110.54, 55.62, 42.54, 41.35, 40.14, 22.55.

Poly(N-isopropylacrylamide)-Poly(N-vinylpyrrolidinone) (IA -VP). Reaction: 24 mg polyIA (0.21 mmol N-isopropylacrylamide residues estimated, 1 equiv) and N-vinylpyrrolidinone 8 (0.455 mL, 4.26 mmol, 20 equiv), heated for 4.5 h (formed a glassy solid/gel). Precipitation: reaction mixture extracted with THF [discarding gelatinous homopolymer polyVP], precipitated with diethyl ether to give polyIA-VP, yield 5%. Mn(CHCl₃)=2,300 and PD=1.78; 1H NMR (CDCl₃): d 6.8-5.7 (br s, NH), 4.1-3.5 (br m), 3.5-3.05 (br s, NCH₂), 2.55-0.95 (br m, includes CH₃); 1H signal integration: 1:1.6 ratio of N-isopropylacrylamide:N -vinylpyrrolidinone residues; 13C NMR (CDCl₃) d 174.49, 44.75, 43.53, 42.81, 41.25, 22.53, 18.49; LCST(H₂₀)=35° C.

Parallel Graft Copolymer synthesis.

Statistical Copolymers. Poly(styrene-stat-4) [S(4)]. A solution of 4 (311 mg, 0.90 mmol, 1 equiv), AIBN (90 mg, 0.55 mmol, 0.6 equiv) and styrene 5 (1.05 mL, 9.2 mmol, 10 equiv) in DCB (3 mL) was freeze thawed three times and then heated to 70 oC for 8 h. Precipitation: DCB/methanol gave polyS(4) as a white solid, yield 92%. Mn(THF)=12,600 and PD=2.84; 1H NMR (CDCl₃) d 7.5-6.7 (br m, Ar-H), 6.7-6.1 (br m, Ar-H), 4.6-4.2 (br s, 2H), 4.0-3.1 (br m), 2.6-0.65 (br m), 0.65-0.0 (br m, TEMPO); 1H signal integration: 11.2:1 ratio of styrene: 4 residues; 13C NMR (CDCl₃) d 176.20, 145.33, 127.96, 127.65, 125.65, 88.55, 65.55, 59.87, 40.39, 33.96, 20.35, 17.15.

Poly(3,4-dimethoxystyrene-stat-4) [DS(4)]. Reaction: 282 mg of 4 (0.82 mmol, 1 equiv), AIBN (92 mg, 0.56 mmol, 0.7 equiv) and 3,4-dimethoxystyrene 7 (1.2 mL, 8.1 mmol, 10 equiv) in DCB (3 mL). Precipitation: THF/methanol, to give polyDS(4) as a white solid, yield 70%. Mn(THF) 18,500 and PD=2.30; 1H NMR (CDCl₃) d 7.3-6.9 (br s, Ar-H), 6.75-6.3 (br m, Ar-H), 6.3-5.75 (br m, Ar-H), 4.69 (br s), 4.42 (br s), 4.0-3.3 (br d, —OCH₃), 2.3-0.8 (br m), 0.8-0.2 (br m); 1H signal integration: 11.0:1 ratio of 3,4-dimethoxystyrene:phenyl (derived from 4) residues; 13C NMR (CDCl₃) d 176.02, 148.41, 147.03, 137.89, 128.07, 124.94, 119.49, 110.58, 83.67, 65.83, 60.19, 55.63, 40.14, 33.92, 20.28, 17.17.

Poly(N-vinylpyrrolidinone-stat-4) [VP(4)]. Reaction: 291 mg of 4 (0.84 mmol, 1 equiv), AIBN (93 mg, 0.57 mmol, 0.7 equiv) and N-vinylpyrrolidinone 8 (0.90 mL, 8.4 mmol, 10 equiv) in DCB (3 mL). Precipitation: THF/diethyl ether to give polyVP(4) as a white solid, yield 90%. Mn(CHCl₃)=65,700 and PD=1.49; 1H NMR (CDCl₃) d 7.3-6.95 (br m, Ar-H), 4.71 (br s), 4.28 (br s), 4.1-3.35 (br m, 1H, NCH), 3.35-2.8 (br s, 2H, NCH₂), 2.8-0.4 (br m); by integration: 4.1:1 ratio of N-vinylpyrrolidinone:phenyl (derived from 4) residues; 13C NMR (CDCl₃) d 175.29, 127.68, 83.46, 66.30, 59.94, 44.72, 43.46, 42.00, 33.94, 31.37, 20.32, 17.06.

2. Graft Copolymers. Poly(styrene-stat-8)-g-raft-poly(3,4-dimethoxystyrene) [S(4)-DS]. Reaction: 109 mg of polyS(4) and 3,4-dimethoxystyrene 7 (1.0 mL). Precipitation: DCM/methanol to give polyS(4)-DS as a white solid, yield 72%. Mn(THF)=94,300 and PD=1.39; 1H NMR (CDCl₃) d 7.2-5.75 (br m), 4.0-3.3 (br d, —OCH₃), 2.3-0.8 (br m); 1H signal integration: 11.0:1 ratio of 3,4-dimethoxystyrene:styrene residues; 13C NMR (CDCl₃) d 148.00, 146.87, 137.67, 127.98, 125.43, 119.38, 110.58, 55.63, 40.18.

Poly(styrene-stat-8)-graft-poly(N-vinylpyrrolidinone) [S(4) -VP]. Reaction: 104 mg of polyS(4) and N-vinylpyrrolidinone 8 (1.0 mL). Precipitation: DCM/diethyl ether to give polyS(4)-VP as a white solid, yield 13%. Mn(CHCl₃)=9,200 and PD=1.84; 1H NMR (CDCl₃) d 7.3-6.2 (br m, Ar-H), 4.1-3.5 (br m, 1H, NCH), 3.5-2.9 (br s, 2H, NCH₂), 2.5-0.85 (br m); 1H signal integration: 1.6:1 ratio of styrene:N-vinylpyrrolidinone residues; 13C NMR (CDCl₃) d 175.40, 145.84, 127.96, 125.67, 60.55, 44.20, 43.42, 42.84, 40.30, 31.45, 18.36.

Poly(3,4-dimethoxystyrene-stat-8)-graft-polystyrene [DS(4) -S]. Reaction: 105 mg of polyDS(4) and styrene 5 (1.0 mL). Precipitation: DCM/methanol to give polyDS(4)-S, yield 49%. Mn(THF)=136,000 and PD=1.42; 1H NMR (CDCl₃) d 7.55-5.8 (br m), 4.0-3.4 (br d, —OCH₃), 2.4-0.9 (br m); 1H signal integration: 9.9:1 ratio of styrene:3,4-dimethoxystyrene residues; 13C NMR (CDCl₃) d 148.65, 147.35, 145.27, 137.41, 127.64, 125.68, 119.19, 111.05, 55.79, 40.43.

Poly(3,4-dimethoxystyrene-stat-8)-graft-poly(N -vinylpyrrolidinone) [DS(4)-VP]. Reaction: 102 mg of polyDS(4) and N-vinylpyrrolidinone 8 (1.0 mL). Precipitation: DCM/diethyl ether to give polyDS(4)-VP as a white solid, yield 18%. Mn(CHCl₃)=12,100 and PD=1.40; 1H NMR (CDCl₃): d=7.3-6.7 (br s, Ar4-H), 6.7-6.3 (br m, ArDS-H), 6.3-5.7 (br m, ArDS-H), 4.1-2.8 (br m), 2.6-0.8 (br m), 0.8-0.1 (br s); by integration: 2.3:1 ratio of N-vinylpyrrolidinone:3,4-dimethoxystyrene residues; 13C NMR (CDCl₃) d 175.23, 148.52, 146.86, 137.89, 119.34, 110.53, 65.78, 55.62, 43.56, 42.47, 40.13, 31.39, 20.29, 18.25, 16.99.

Verification of Block Copolymer Structures Derived from Initiator 2 via Saponification (FIG. 2). The general procedure for the synthesis and verification of the block copolymer structure is a three step process. Following two rounds of polymerizations the ester linker between the polymer blocks is cleaved by saponification. SEC analysis occurs after each of the three stages. The generation of a block copolymer of polystyrene (polyS-S) as detailed below is illustrative.

First polymerization: A solution of initiator 100 (224 mg, 0.28 mmol, 1 equiv; 200, 3, 4 can be used in lieu of 100) and styrene 5 (0.65 mL, 5.7 mmol, 20 equiv) in DCB (3 mL) was degassed by 3 cycles of freezing/thawing under vacuum then heated at 70° C. under nitrogen with mixing for 18 h. The reaction mixture was precipitated into hexane, dissolved in DCM, precipitated into methanol, and dried to give polys as a white solid (392 mg, 66%). Mn(THF)=8200 and PD=1.69.

Second polymerization: The homo polymer polyS obtained from the first polymerization (20 mg, 0.19 mmol styrene residues estimated, 1 equiv) was dissolved in styrene 5 (1.09 mL, 9.5 mmol, 50 equiv), degassed as described vide supra, and then heated at 130° C. for 18 h. The reaction mixture then was diluted and precipitated twice (DCM/methanol) to give the block copolymer polyS-S as a white solid (619 mg, 61%). Mn(THF)=264,000 and PD=1.30.

Ester hydrolysis: PolyS-S (15 mg) was dissolved in THF (4 mL) and mixed with methanol (1 mL) and 2 N NaOH (1 mL) forming an emulsion that was rapidly stirred at room temperature. After 3 h, stirring was stopped and phase separation was assisted by addition of water (2 mL) and diethyl ether (2 mL). A portion of the organic phase was mixed with a small sample of the thin layer of emulsion at the interface of phases, evaporated to dryness, dissolved in THF, and analyzed by SEC (Mn(THF)=118,000 and PD=1.22; Mn(THF)=8700 and PD=1.44).

Synthesis of Block Copolymer polyIA-S for TEM Analysis First polymerization: 92 mg of 200 (0.12 mmol, 1 equiv) and N-isopropylacrylamide 9 (3.38 g, 30 mmol, 250 equiv) were dissolved in DMF (10 mL), and polymerization was performed as described vide supra. Precipitation occurred from THF/diethyl ether to give polyIA as a white solid, yield 88%. Mn(CHCl₃)=9300 and PD=1.40; 1H NMR (CDCl₃) d 7.0-6.2 (br s, 1H, NH), 3.95 (br s, 1H, NCH), 2.4-1.25 (br m), 1.25-0.9 (br s, 6H, CH₃); 13C NMR (CDCl₃) d 174.62, 42.34, 41.28, 22.53.

Second polymerization: 1.12 g polyIA from the first polymerization (9.9 mmol N-isopropylacrylamide residues estimated, 1 equiv) was dissolved first in 1.4 mL DMF with gentle warming, and styrene 5 (2.26 mL, 19.7 mmol, 2 equiv) was added. The second polymerization then was performed as described vide supra. Precipitation occurred with DCM/methanol followed by DCM/diethyl ether to give polyIA-S as a white solid, yield 22%; 1H NMR (CDCl₃) d 7.4-6.25 (br m, Ar-H), 4.00 (br s, —NCH—), 2.4-0.95 (br m, includes —CH₃); 1H signal integration: 3.2:1 ratio of styrene:N-isopropylacrylamide residues.

Solid-liquid extraction: polyIA-S (594 mg) was placed into a Soxhlet extractor, extracted with diethyl ether (17 h), dried, and then extracted with methanol (23 h), and dried. Yield 515 mg of a white solid (87%). Mn(THF)=145,000 and PD=1.28; 1H signal integration: 4.1:1 ratio of styrene:N-isopropylacrylamide residues. 13C NMR (CDCl₃) d 174.53, 145.08, 127.41, 125.62, 42.37, 41.28, 40.33, 22.60.

Control Polymerization with AIBN Initiator. First polymerization: 21 mg of AIBN (0.13 mmol, 1 equiv) and N-isopropylacrylamide 9 (3.46 g, 30.6 mmol, 240 equiv) in DMF (10 mL). Precipitation: THF/diethyl ether to give polylA as a white solid, yield 96%; 1H NMR (CDCl₃): d=6.9-6.1 (br s, 1H, —NH), 3.92 (br s, 1H, —NCH—), 2.35-1.2 (br m), 1.2-0.9 (br s, 6H, —CH₃). Second polymerization: 1.113 g poly(N-isopropylacrylamide) (9.8 mmol N-isopropylacrylamide residues estimated, 1 equiv) dissolved first in 1.4 mL DMF with gentle warming, styrene 5 (2.26 mL, 19.7 mmol, 2 equiv). Precipitation: DCM/methanol, then DCM/diethyl ether, yield 3%; Mn(THF)=386,000 and PD=1.68; 1H NMR (CDCl₃) d=7.45-6.85 (br m, Ar-H), 6.85-6.25 (br m, Ar-H), 4.1-3.9 (br s, 1H, —NCH—), 2.4-1.25 (br m, aliphatic polymer backbone), 1.25-1.05 (br s, 6H, —CH₃); 1H signal integration: 16:1 ratio of styrene:N -isopropylacrylamide residues.13C NMR (CDCl₃) d 175.30, 145.32, 127.94, 125.66, 42.90, 41.61, 40.37, 22.66.

Methods incorporating polyBS-DS into LPOS.

Reduction of a-Nitriles. a) Metal hydride reduction to form polyBS-DS-NH_{2 22}. LiAlH₄ (0.51 g, 13.4 mmol, 76 equiv) was added portionwise to copolymer polyBS-DS (1.5 g, Mn(THF)=17,000 and PD=2.45; 0.088 mmol 2 equiv) dissolved in THF (100 mL) and heated to reflux for 2 h. After cooling and quenching carefully with water (1 mL) and 1 N NaOH (1 mL), the reaction mixture was filtered twice through celite, concentrated (ca. 2 mL), precipitated into methanol (50 mL), and dried to give 22 as a white solid, yield 83%; Mn(CHCl₃)=16,400 and PD=1.58; 1H NMR (CDCl₃) unchanged from polyBS-DS reported vide supra. CH₂NH₂ resonances overlap with those of polymer backbone; quantitative ninhydrin: 0.14 mmol amine per gram polymer (ninhydrin assay was negative for polyBS-DS prior to LiAlH₄ treatment).

b) Hydrogenation. A homopolymer of polys (derived from 100, 2.0 g, Mn(CHCl₃)=8400 and PD=1.98; 0.24 mmol estimated from SEC, 2 equiv) was dissolved in dioxane (50 mL) in a Parr bottle. After adding PtO₂ (0.25 g, 1.1 mmol) and CHCl₃ (1 mL), the solution was degassed by bubbling with N2 and then shaken overnight under a H₂ atmosphere (40 psi). The catalyst was removed by filtration through Celite, and the filtrate concentrated (ca. 8 mL). The polymer product was precipitated into methanol (200 mL), and dried to a white solid, yield 89%. Mn(CHCl₃)=8500 and PD=1.79; 1H NMR (CDCl₃) unchanged (CH₂NH₂ resonances overlap with those of polymer backbone); quantitative ninhydrin: 0.21 mmol amine per gram polymer (ninhydrin assay was negative for polys prior to LiAlH₄ treatment).

Kinetics of Imine Formation. Copolymer amine 22 from above and 1-aminohexane were prepared as a series of solutions of varying concentrations (30, 20, 10 mM) in CHCl₃ and equimolar 4-dimethylaminocinnamaldehyde 23 was added. The mixture was stirred over a small amount of Na2SO₄ at room temperature. Periodically, aliquots (10 mL) were removed, diluted to 20 mM with 150 mM trifluoroacetic acid in CHCl₃, and the absorbance measured at 466 nm (e466,polymeric Schiff base=63,100; e466,Schiff base of 1-aminohexane=79,000). A plot of x/[a(a−x)] versus time where x=concentration of Schiff base and a=initial concentration of amine gave a straight line indicative of second-order kinetics with the rate constant equal to the slope (J. J. Maher, M. E. Furey, L. J. Greenberg, Tetrahedron Lett. 1971, 27).

Preparation of polyBS-DS supported chiral diphosphine ligand 27. (2S,4S)-1-Glutaroyl-4-diphenylphosphino-2-(diphenylphosphinomethyl)-pyrrolidine 26 as shown in FIG. 5. A solution of (2S, 4S)-4-diphenylphosphino-2-(diphenylphosphino)methyl pyrrolidine 25 (58 mg, 0.13 mmol), glutaric anhydride (19 mg, 0.16 mmol), diisopropylethylamine (DIPEA, 58 mg, 0.33 mmol) and dimethylaminopyridine (DMAP, 1.6 mg, 0.013 mmol) in degassed DCM (1.0 mL) was stirred under an argon atmosphere at room temp (8 h). The reaction mixture then was concentrated in vacuo and applied to a Kieselghur 1 mm preparative TLC plate. The product 26 was isolated as a colorless oil (57 mg, 78%): RF=0.4 (95:5 DCM/MeOH with 2% AcOH); 1H NMR (250 MHz, CDCl₃) d 1.2-1.5 (m, 1H), 2.0-2,4 (m, 10 -H), 2.6-3.1 (m. 6H), 7.0-7.7 (m, 20H); HRFABMS calcd for C₃₄H₃₆NO₃P2 568.2092, obsd 568.2094.

Polymer supported phosphine ligand 27 as shown in FIG. 5. A solution of carboxy-amide 26 (35 mg, 62 mmol) EDC (30 mg, 152 mmol), DMAP (13 mg, 106 mmol) and polyBS-DS-NH_{2 22} (0.14 mmolg-1 amino groups, 135 mg) in degassed DCM, was stirred at room temperature for 8 h or until quantitative ninhydrin analysis was negative. The reaction mixture was then added dropwise into cold MeOH (50 ml) and the precipitate collected by filtration, redissolved in DCM and reprecipitated by addition into MeOH. The precipitate was collected by filtration to give 27 as a free flowing white powder, yield 99%. 1H NMR (CDCl₃) d 7.35-5.75 (br m, Ar-H (masks phenyl protons of ligand), 3.95-3.4 (br d, —OCH₃), 3.0-2.9 (m. ligand protons), 2.2-1.1 (br m, includes t-butyl group).

Catalytic Hydrogenation with 27 as shown in FIG. 5. To an argon purged flask was added the polymer-supported ligand 27 (126 mg, 0.14 mmol of diphosphine per gram of polymer), m-dichloro-bis (1,5-cyclooctadiene)dirhodium(I) (4 mg, 0.008 mmol), and degassed THF (5 ml). The homogeneous mixture was stirred for 4 h and then evaporated under argon and resuspended in degassed DCM (1.5 ml). The rhodium-supported polymer, Rh(I)-27, was then precipitated by dropwise addition into cold, degassed, anhydrous methanol (50 ml). The polymer (pale yellow) was recovered by filtration and dried in vacuo. The Rh(I)-27 complex was then dissolved in degassed THF (10 ml) and 2-N-acetamidoacrylic acid 28 (52 mg, 0.4 mmol) added. The reaction was stirred under H₂ (20psi). After 2 d, the reaction mixture was evaporated to dryness, dissolved in DCM (2 ml) and precipitated as described above. The polymer was recovered by filtration (126 mg, 100%) and the methanolic mother liquor was evaporated to dryness and the products were analyzed by 1H NMR. The ratio of 1H NMR integrations between N-acetyl alanine 29 (CD30D, d 1.99) and starting material 28 (CD30D, d 2.06) N -acetyl peaks was used to determine conversion=50% after 2.5 d. No attempt to optimize this reaction was made.

Catalytic Hydrogenation with Soluble Ligand: (2,S, 4,S)-1-tert-butoxycarbonyl-4-diphenyl-phosphino-2-(diphenylphosphinomethyl)pyrrolidine. The method and relative equivalents of all the reagents is as described above for 27. Conversion (as determined by 1H NMR)=40% after 2.5 d. No attempt to optimize this reaction was made.

Enantiomeric Excess Determination. The reaction products from the catalytic hydrogenations with either polymer supported ligand 27 or the soluble ligand vide supra were dissolved in DCM (5 mL), and (R)-(+) 1-(naphthyl)ethylamine (12 mg, 66 mmol), EDC (13 mg, 70 mmol) and DMAP (8.5 mg, 70 mmol) were added. The reaction mixtures were stirred at room temperature (2 h). The crude reaction mixtures were then analyzed by HPLC [mobile phase 30:70 acetonitrile water (0.1% TFA)] RT (S)-29=43.01 RT (S)-29=43.73.

Synthesis of NBoc Block Copolymer Supports with Initiator 3.

1. polyBS-DS-(NBoc). First polymerization: 101 mg of 3 (0.098 mmol, 1 equiv) and 4-tert-butylstyrene 6 (0.36 mL, 1.97 mmol, 20 equiv) in DCB (1 mL). Precipitation: DCM/methanol to give polyBS as a white solid, yield 81%; Mn(THF)=5,000 and PD =2.43; 1H NMR (CDCl₃) d 7.5-6.8 (br m, Ar-H), 6.8-6.0 (br m, Ar-H), 4.87 (br d, CH₃), 4.57 (br d, CH₃), 4.25 (br m, CH₃), 3.76 (br s, CH₃), 2.5-1.6 (br m), 1.42 (br s, t-butylBoc), 1.27 (br s,t-butylBs), 0.88 (br s), 0.66 (br d); 1H signal integration: 15:1 ratio of 4-tert-butylstyrene:phenyl (derived from 4) residues; 13C NMR (CDCl₃) d 171.67, 148.13, 142.91, 128.47, 127.65, 124.88, 83.96, 66.07, 60.12, 46.34, 39.57, 34.02, 33.58, 31.58, 28.13, 20.51, 19.11. Second polymerization: 209 mg polyBS derived from 3 (1.3 mmol 4-tert -butylstyrene residues estimated, 1 equiv) dissolved in 3,4-dimethoxystyrene 7 (0.21 mL, 1.4 mmol, 1.1 equiv). Precipitation: DCM/methanol to give polyBS-DS-(NBoc) as a white solid, yield 74%. Mn(CHCl₃)=24,300 and PD=1.87; 1H NMR (CDCl₃) d 7.3-6.8 (br m, Ar-H), 6.8-5.75 (br m, Ar-H), 3.95-3.4 (br d, —OCH₃), 2.5-0.2 (br m), 1.43 (br s, t-butylBoc), 1.27 (br s,t-butylBs); 1H signal integration: 1.2:1 ratio of 4-tert -butylstyrene:3,4-dimethoxystyrene residues; 13C NMR (CDCl₃) d 148.79, 148.04, 147.13, 142.85, 137.34, 127.54, 124.84, 119.82, 110.77, 55.47, 39.90, 38.53, 33.95, 31.19.

2. polyVP-S-(NBoc). First polymerization: 100 mg of 3 (0.097 mmol, 1 equiv) and N-vinylpyrrolidinone 8 (0.21 mL, 1.97 mmol, 20 equiv) in DCB (1 mL). Precipitation: DCB/diethyl ether to give polyVP as a white solid, yield 78%. Mn(CHCl₃)=33,200 and PD=1.66; 1H NMR (CDCl₃) d 7.23 (br s, Ar-H), 4.82 (br s, CH₃), 4.51 (br s, CH₃), 4.23 (br s, CH₃), 4.1-3.45 (br m, NCH), 3.45-2.85 (br s, NCH₂), 2.6-1.4 (br m), 1.35 (br s, t -butylBoc), 1.27, 1.20, 1.04, 0.60 (each br s, CH₂ and/or CH₃ of TEMPO); 1H signal integration: 27:1 ratio of N -vinylpyrrolidinone:phenyl (derived from 3) residues; 13C NMR (CDCl₃) d 175.27, 171.53, 128.06, 127.58, 83.84, 65.76, 60.39, 46.45, 44.75, 43.47, 41.99, 33.76, 31.40, 28.34, 18.24. Second polymerization: 54 mg polyVP derived from 3 (0.49 mmol N -vinylpyrrolidinone residues estimated, 1 equiv) dissolved first in 0.4 mL DMF with gentle warming, styrene 5 (1.0 mL, 8.7 mmol, 18 equiv). Precipitation: DCM/methanol to give polyVP-S-(NBoc) as a white solid, yield 53%. Mn(THF)=48,800 and PD=1.41; 1H NMR (CDCl₃) d=7.4-6.9 (br m, Ar-H), 6.9-6.3 (br m, Ar-H), 4.15-3.55 (br m, NCH), 3.55-3.05 (br s, NCH₂), 2.8-0.9 (br m); 1H signal integration: 10.9:1 ratio of styrene:N -vinylpyrrolidinone residues; 13C NMR (CDCl₃) d 175.48, 145.36, 127.47, 125.54, 44.84, 43.91, 42.73, 40.39, 31.47, 18.40.

Boc Deprotection of polyBS-DS-(NBoc). polyBS-DS-(NBoc) (92 mg) was dissolved in DCM (0.25 mL) and trifluoroacetic acid (TFA) (25 mL) was added. After stirring for 15 h, the volatiles were evaporated under a stream of N2, the residue was dissolved in DCM (1 mL) and washed with 1 N NaHCO₃ (3×1 mL), brine (1 mL). After drying over Na2SO₄, the polymer solution was concentrated and precipitation induced by dropwise addition to methanol. The filtrate was collected to give deprotected polyBS-DS-(NBoc) as a white solid, yield 81%. Mn(CHCl₃)=20,400 and PD=2.00; 1H NMR (CDCl₃): d 7.35-5.75 (br m, Ar-H), 3.95-3.45 (br d, —OCH₃), 2.25-0.65 (br m), 1.28 (br s,t -butylBs); by integration: 1:1 ratio of 4-tert-butylstyrene:3,4-dimethoxystyrene residues; 13C NMR (CDCl₃): d=148.27, 147.97, 147.08, 142.71, 137.97, 127.13, 124.60, 119.45, 110.52, 55.66, 40.19, 39.79, 34.26, 31.52.

Deprotection of polyVP-S-(NBoc). PolyVP-S-(NBoc) (92 mg) was dissolved in dry DCM (0.25 mL) and TFA (25 mL) added. After stirring for 15 h, the reaction was worked up as above to give Boc deprotected polyVP-S as a white solid, yield 64%. Mn(THF)=52,200 and PD=1.43; 1H NMR (CDCl₃) d 7.35-6.85 (br m, Ar-H), 6.85-6.3 (br m, Ar-H), 4.1-3.5 (br m, NCH), 3.5-3.05 (br s, NCH₂), 2.5-0.9 (br m); 1H signal integration: 10.6:1 ratio of styrene:N-vinylpyrrolidinone residues; 13C NMR (CDCl₃) d 175.43, 145.30, 127.97, 125.66, 44.82, 43.46, 42.26, 40.34, 31.39, 18.32.

Synthesis of Copolymers for “Oscillating Liquid-Phase” (OLP) Synthesis (FIG. 6). (a) organic-Aqueous-Organic. First polymerization: 900 mg of 100 (1.1 mmol, 1 equiv) and N-tert -butylacrylamide 31 (1.43 g, 11 mmol, 10 equiv) in DMF (5 mL). Precipitation: THF/water, then purified through a short bed of silica (95:5 DCM:methanol). Yield 42%; Mn(CHCl₃)=32,100 and PD =2.44; 1H NMR (CDCl₃) d 7.2 (br m, Ar-H), 4.85 (br s), 4.70 (br s), 4.53 (br s), 4.23 (br s), 2.3-1.4 (br m), 1.4-1.1 (br s, t -butyl group), 0.94 (br s), 0.63 (br s); 13C NMR (CDCl₃) d 174.99, 127.98, 127.60, 83.71, 42.78, 40.32, 36.47, 33.95, 20.34, 17.02. Second polymerization: 58 mg polyBA (0.46 mmol N-tert-butylacrylamide residues estimated, 1 equiv) dissolved first in 0.45 mL DMF, acrylamide 32 (327 mg, 4.6 mmol, 10 equiv). Precipitation: water/methanol. Yield 21%; (incompatibility of SEC column with aqueous solvents precluded analysis); 1H NMR (D20) d 2.4-1.4 (br m), 1.3 (br s, t-butyl group); 1H signal integration: 1:140 ratio of N-tert -butylacrylamide:acrylamide residues; 13C NMR (D20) d=181.79, 44.08, 37.63, 36.77, 28.83. Ester hydrolysis: 42.5 mg copolymer polyBA-AA stirred with 1 N NaOH (5 mL) for 7 d. Extraction with ethyl acetate gave polyBA as a white solid, yield 89% [based on the weight of polyBA contained in block copolymer polyBA-AA (estimated from 1H NMR integration)]; Mn(CHCl₃)=46,500 and PD=2.29; 1H NMR (CDCl₃): d=2.3-1.45 (br m), 1.45-1.15 (br s, t-butyl group).

(b) Aqueous-organic-Aqueous. First polymerization: 146 mg of 2 (0.18 mmol, 1 equiv) and N-vinylpyrrolidinone 8 (0.39 mL, 3.6 mmol, 20 equiv) in DCB (1.5 mL). Precipitation: THF/diethyl ether to give a white solid, yield 77%; Mn(CHCl₃) 1,100 and PD=1.46; 1H NMR (CDCl₃) d 7.3-6.9 (br m, Ar-H), 4.80 (br s), 4.62 (br s), 4.49 (br s), 4.3-3.35 (br m, 1H, NCH), 3.35-2.8 (br s, 2H, NCH₂), 2.55-1.15 (br m), 1.06 (br s), 0.91 (br s), 0.57 (br s); 1H signal integration: 15:1 ratio of N -vinylpyrrolidinone:phenyl (derived from 2) residues; 13C NMR (CDCl₃): d=175.34, 127.92, 127.56, 83.61, 44.79, 43.54, 42.32, 40.27, 33.84, 31.33, 20.15, 18.18, 16.98. Second polymerization: 54 mg polyVP (0.49 mmol N-vinylpyrrolidinone residues estimated, 1 equiv) dissolved first in 0.36 mL DMF, then 4-tert-butylstyrene 6 (0.89 mL, 4.9 mmol, 10 equiv). Precipitation: DCM/methanol to give a white solid, yield 59%. Mn(THF)=129,000 and PD=1.66; 1H NMR (CDCl₃) d 7.35-6.8 (br m, Ar-H), 6.8-6.05 (br m, Ar-H), 4.05-3.5 (br m, NCH), 3.5-3.05 (br s, NCH₂), 2.55-1.1 (br m, includes t-butyl group); 1H signal integration: 1:8 ratio of N-vinylpyrrolidinone:4-tert -butylstyrene residues; 13C NMR (CDCl₃) d 174.30, 147.98, 142.71, 127.19, 124.59, 45.05, 43.53, 42.56, 39.75, 34.25, 31.51, 18.28. Ester hydrolysis: 105 mg copolymer polyVP-BS dissolved in THF (6 mL) mixed with a solution of KCN (16 mg) in methanol (3 mL), overnight. Evaporated solvents, dissolved/slurried solids in CHCl₃ (0.5 mL), precipitated polyBS by addition of methanol (5 ml) and isolated by filtration. polyVP was recovered from the filtrate, yield 32% (based on weight of polyN-vinylpyrrolidinone contained in block copolymer estimated from 1H NMR integration); Mn(CHCl₃)=1,000 and PD=1.90; 1H NMR (CDCl₃): d=4.05-3.5 (br m, 1H, —NCH—), 3.5-3.05 (br s, 2H, —NCH₂—), 2.55-1.3 (br m).

What is claimed is:

1. A process for transferring a chemical intermediate from a first solvent to a second solvent, the first and second solvents being immiscible with one another, the process comprising the following steps:
Step A: providing a first conjugate as a solute in said first solvent, said first conjugate including said chemical intermediate, a platform, and a first carrier, said chemical intermediate and said first carrier being attached to said platform, said first carrier having a solubility in said first solvent for imparting solubility to said first conjugate in said first solvent; then
Step B: converting said first conjugate into a second conjugate, said second conjugate including said first conjugate and a second carrier, said second carrier being attached to said platform and having a solubility in said second solvent for imparting solubility to said second conjugate in said second solvent; and
Step C: contacting said second conjugate with said second solvent for transferring said second conjugate together with said chemical intermediate attached thereto from said first solvent into said second solvent.

2. A process for transferring a chemical intermediate from a second solvent to a first solvent, the first and second solvents being immiscible with one another, the process comprising the following steps:
Step A: providing a second conjugate as a solute in said second solvent, said second conjugate including said chemical intermediate, a platform, a first carrier, and a second carrier, said chemical intermediate, said first carrier and said second carrier being attached to said platform, said second carrier having a solubility in said second solvent for imparting solubility to said second conjugate in said second solvent; then
Step B: cleaving the second carrier from said platform of the second conjugate of said Step A for transferring the chemical intermediate from the second solvent to the first solvent by forming a first conjugate having a solubility in said first solvent.

3. A process for transferring a chemical intermediate from a first solvent to a second solvent and then from said second solvent to said first solvent, the first and second solvents being immiscible with one another, the process comprising the following steps:
Step A: providing a first conjugate as a solute in said first solvent, said first conjugate including said chemical intermediate, a platform, and a first carrier, said chemical intermediate and said first carrier being attached to said platform, said first carrier having a solubility in said first solvent for imparting solubility to said first conjugate in said first solvent; then
Step B: converting said first conjugate into a second conjugate, said second conjugate including said first conjugate and a second carrier, said second carrier being attached to said platform and having a solubility in said second solvent for imparting solubility to said second conjugate in said second solvent; and
Step C: contacting said second conjugate with said second solvent for transferring said second conjugate together with said chemical intermediate attached thereto from said first solvent into said second solvent.
Step D: cleaving the second carrier from said platform of the second conjugate of said Step B for transferring the chemical intermediate from the first solvent to the second solvent and then from said second solvent to said frist solvent, by reforming the first conjugate having a solubility in said first solvent.

4. A process for transferring a chemical intermediate into a first solvent, the process comprising the following steps:
Step A: conjugating said chemical intermediate and a first carrier to a platform for forming a first conjugate; and then
Step B: contacting said first conjugate with said first solvent for transferring said first conjugate together with first chemical intermediate attached thereto into said first solvent.

5. A process for converting a first chemical intermediate into a second and third chemical intermediate, the process comprising the following steps:
Step A: providing a first conjugate as a solute in said first solvent, said first conjugate including said first chemical intermediate, a platform, and a first carrier, said chemical intermediate and said first carrier being attached to said platform, said first carrier having a solubility in said first solvent for imparting solubility to said first conjugate in said first solvent; then
Step B: converting said first chemical intermediate attached to said first conjugate into said second chemical intermediate in said first solvent for forming a modified first conjugate; then
Step C: converting said modified first conjugate having said second chemical intermediate attached thereto into a second conjugate, said second conjugate including said second chemical intermediate, said platform, said first carrier, and said second carrier, said second chemical intermediate and said first and second carriers being attached to said platform, said second carrier having a solubility in said second solvent for imparting solubility to said second conjugate in said second solvent;
Step D: contacting said second conjugate with said second solvent for transferring said second conjugate together with said second chemical intermediate attached thereto into said second solvent; and then
Step E: converting said second chemical intermediate attached to said second conjugate into said third chemical intermediate in said second solvent for forming a modified second conjugate.

6. A process for synthesizing a first conjugate including a platform and a first carrier, said first carrier being attached to said platform, the process comprising the following steps:
Step A: providing a bifunctional initiator and a first carrier; then
Step B: converting the bifunctional initiator and the first carrier of said Step A into the first conjugate by heating said bifunctional intiator and said first carrier, said bifunctional initiator converting into said platform and wherein said first carrier being attached to said platform for synthesizing the first conjugate.

7. A process for synthesizing a second conjugate including a platform, a first carrier, and a second carrier, said first and second carriers being attached to said platform, the process comprising the following steps:
Step A: providing a first conjugate as a solute in a first solvent, said first conjugate including a platform and said first carrier, said first carrier being attached to said platform, said first carrier having a solubility in said first solvent for imparting solubility to said first conjugate in said first solvent; then
Step B: converting said first conjugate into a second conjugate, said second conjugate including said first conjugate and a second carrier, said second carrier being attached to said platform and having a solubility in a second solvent for imparting solubility to said second conjugate in said second solvent.

8. A composition comprising a solution including a first solvent and a first conjugate mixed as a solute therein, said first conjugate including a chemical intermediate, a platform, and a first carrier, said chemical intermediate and said first carrier being attached to said platform.

9. A composition comprising a solution including a second solvent and a second conjugate mixed as a solute therein, said second conjugate including a chemical intermediate, a platform, a first carrier and a second carrier, said chemical intermediate, said first carrier and said second carrier being attached to said platform.

10. A composition comprising a solution including a first solvent and a first conjugate mixed as a solute therein, said first conjugate including a chemical intermediate, a platform, and a plurality of a first carrier, said chemical intermediate and said plurality of the first carrier being attached to said platform.

11. A first conjugate represented by the following structure:

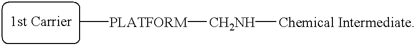

12. A second conjugate represented by the following structure:

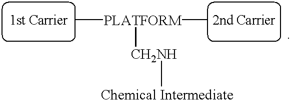

13. An advanced intermediate represented by the following structure:

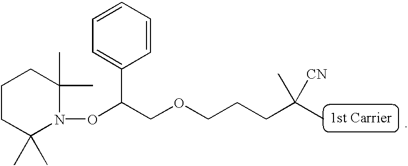

14. A first conjugate represented by one of the following structure:

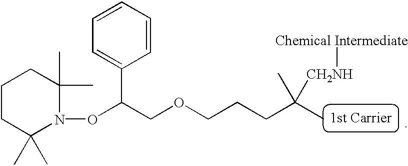

15. A second conjugate represented by the following structure:

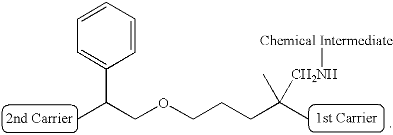

16. An advanced intermediate represented by the following structure:

[chemical structure]

17. A first conjugate represented by the following structure:

[chemical structure]

18. A second conjugate represented by the following structure:

[chemical structure]

19. A bifunctional initiator represented by the following structure:

[chemical structure]

20. A bifunctional initiator represented by the following structure:

[chemical structure]

21. A bifunctional initiator represented by the following structure:

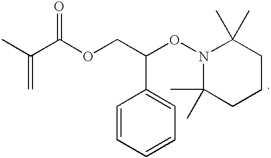

22. A conjugate molecule represented by the following structure: [chemical structure]
